(19) United States
(12) Patent Application Publication
(10) Pub. No.: US 2026/0250300 A1
(43) Pub. Date: Aug. 27, 2026

(54) POLYMORPHS AND THEIR USE FOR TREATING CANCER

(71) Applicant: Cairn Therapeutics, Inc., Wilmington, DE (US)

(72) Inventors: Roger A. RAJEWSKI, Lawrence, KS (US); Nico SETIAWAN, Lafayette, IN (US); Lisa MCCRACKEN, Lafayette, IN (US)

(21) Appl. No.: 19/416,929

(22) Filed: Dec. 11, 2025

Related U.S. Application Data
(63) Continuation of application No. PCT/US2024/033515, filed on Jun. 12, 2024.
(60) Provisional application No. 63/507,954, filed on Jun. 13, 2023.

(51) Int. Cl. C07D 498/06; A61K 31/5365

(52) U.S. Cl. CPC C07D 498/06; A61K 31/5365

(57) ABSTRACT

The present disclosure relates to novel crystalline polymorphic forms of Compound 1, and to processes for their preparation and use. Such polymorphic forms may be a component of a pharmaceutical composition and may be used to treat cancer.

### CROSS-REFERENCE TO RELATED APPLICATIONS

This application claims priority to, and the benefit of, U.S. provisional patent application No. 63/507,954, filed Jun. 13, 2023, the disclosure of which is incorporated herein by reference in its entirety.

### FIELD

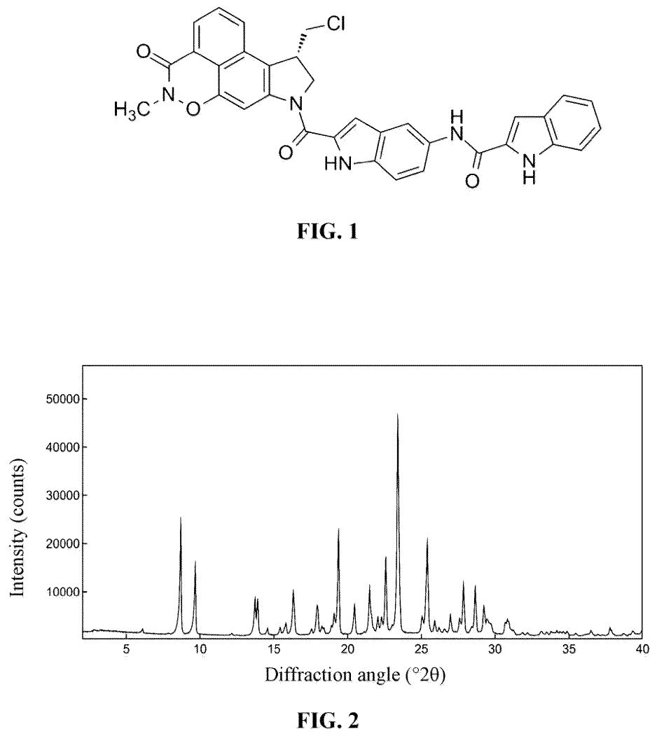

The present disclosure relates to novel polymorphic forms of Compound 1 having the structure depicted in FIG. 1 and the chemical name(S)—N-(2-(10-(Chloromethyl)-5-methyl-4-oxo-5,8,9,10-tetrahydro-4H-pyrrolo[3′,2′: 5,6]naphtho[1,8-de][1,2]oxazine-8-carbonyl)-1H-indol-5-yl)-1H-indole-2-carboxamide. The disclosure is also directed to pharmaceutical compositions comprising a polymorphic form of Compound 1 and to therapeutic and/or prophylactic uses of such polymorphic forms and compositions. Compound 1 and methods of making it are described in U.S. Pat. No. 9,586,974, the disclosure of which is herein incorporated by reference in its entirety.

### BACKGROUND

Approximately 610,000 people die in the United States each year from cancer. Worldwide, there are approximately 10 million cancer deaths each year, accounting for about one in six deaths. Despite decades of research in the field of cancer prevention and treatment, the number of deaths from cancer is increasing from year to year, and many cancers either do not respond to, or develop resistance to, traditional chemotherapies and other therapeutics.

There remains a need for novel compounds for use in the treatment of cancer.

### SUMMARY

Provided herein are methods of making a Compound 1 crystalline Form A hydrate.

Provided herein are methods for making a Compound 1 crystalline Form B DMF solvate.

Provided herein are methods for making a Compound 1 crystalline Form C anhydrate.

Provided herein are methods for making a Compound 1 crystalline Form E DMSO solvate.

Provided herein are methods for making a Compound 1 crystalline Form F DMA solvate.

Provided herein is a Compound 1 crystalline Form A hydrate that is substantially free of Compound 1 crystalline Form C anhydrate.

Provided herein is a Compound 1 crystalline Form C anhydrate that is substantially free of Compound 1 crystalline Form A hydrate.

Provided herein is a Compound 1 crystalline Form A hydrate, further characterized by at a temperature of about 40° C., in absolute ethanol for about two days, the Compound 1 crystalline Form A hydrate converts to the crystalline Form C anhydrate.

Provided herein is a Compound 1 crystalline Form A hydrate, further characterized by at a temperature of about 50° C., in absolute ethanol for about one hour, the Compound 1 crystalline Form A hydrate converts to the crystalline Form C anhydrate.

Further provided are methods for making Compound 1 crystalline Form C anhydrate, comprising heating a solution of Compound 1 crystalline Form A hydrate in absolute ethanol at a temperature of about 40° C. for about two days to provide Compound 1 crystalline Form C anhydrate.

In some embodiments, provided herein is a method for treating cancer in a mammal in need thereof, comprising the step of administering to the mammal an effective amount of any of Compound 1 crystalline Forms A-F.

Provided herein is a Compound 1 crystalline Form A hydrate.

Provided herein is a Compound 1 crystalline Form B DMF solvate.

Provided herein is a Compound 1 crystalline Form C anhydrate.

Provided herein is a Compound 1 crystalline Form E DMSO solvate.

Provided herein is a Compound 1 crystalline Form F DMA solvate.

Provided herein are compositions a) comprising the Compound 1 crystalline Form A hydrate or b) in which the Compound 1 crystalline Form A hydrate is dissolved.

Provided herein is a composition a) comprising the Compound 1 crystalline Form B DMF solvate or b) in which the Compound 1 crystalline Form B DMF solvate is dissolved.

Provided herein are compositions a) comprising the Compound 1 Form C anhydrate or b) in which the Compound 1 Form C anhydrate is dissolved.

Provided herein are compositions a) comprising the Compound 1 Form E DMSO solvate or b) in which the Compound 1 Form E DMSO solvate is dissolved.

Provided herein are compositions a) comprising the Compound 1 Form F DMA solvate or b) in which the Compound 1 Form F DMA solvate is dissolved.

Provided herein are methods of making Compound 1 crystalline Form C anhydrate, the method comprising admixing absolute ethanol and crystalline Form A hydrate to form a slurry, wherein the slurry comprises the crystalline Form C anhydrate.

### BRIEF DESCRIPTION OF THE DRA WINGS

FIG. 1 shows the structure of Compound 1.

FIG. 2 shows a powder X-ray diffraction pattern diffractogram of Compound 1 crystalline Form A hydrate.

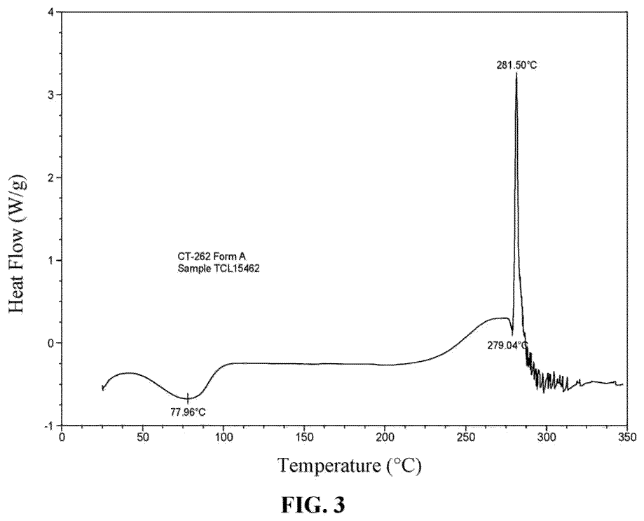

FIG. 3 shows a differential scanning calorimetry curve of Compound 1 crystalline Form A hydrate.

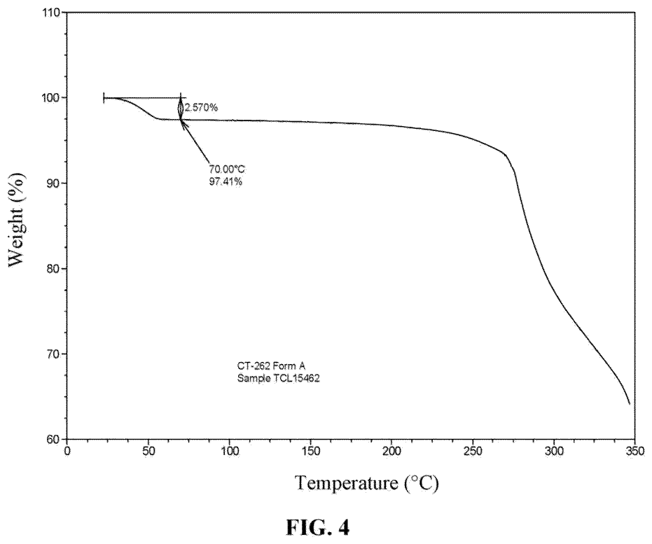

FIG. 4 shows a thermogravimetric analysis curve of Compound 1 crystalline Form A hydrate.

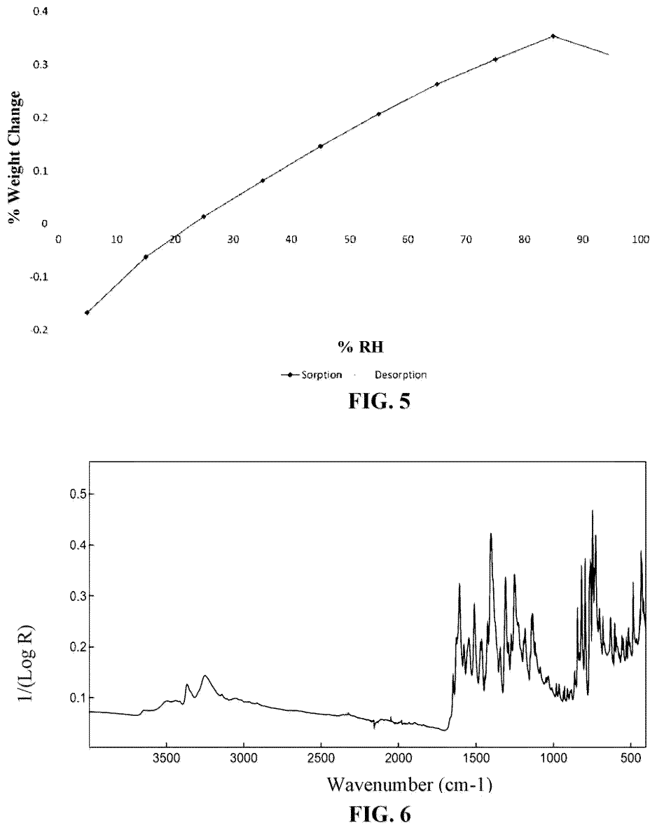

FIG. 5 shows a dynamic vapor sorption analysis of Compound 1 crystalline Form A hydrate.

FIG. 6 shows an infrared spectrum of Compound 1 crystalline Form A hydrate.

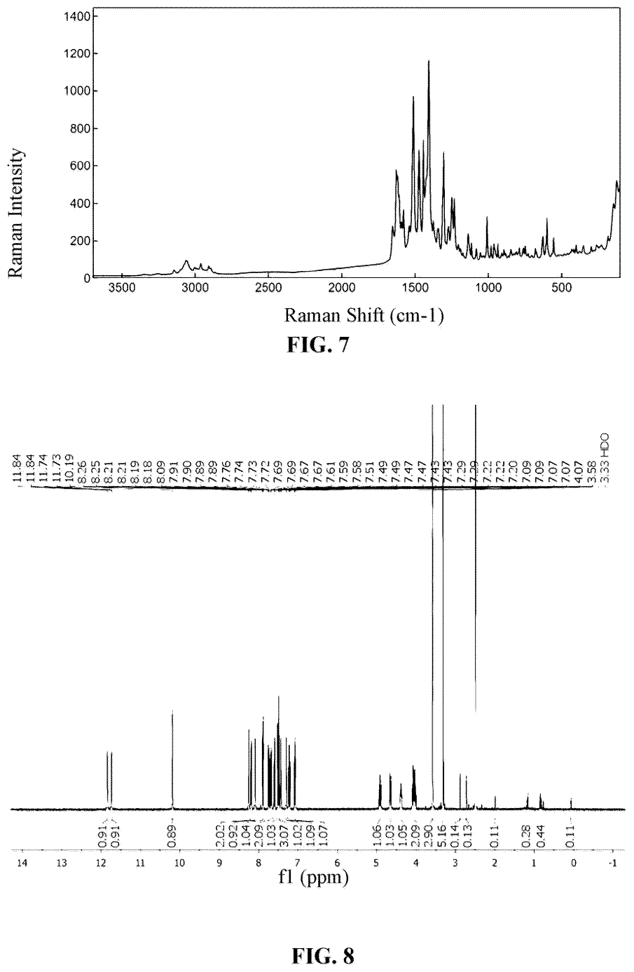

FIG. 7 shows a Raman spectrum of Compound 1 crystalline Form A hydrate.

FIG. 8 shows a ¹H nuclear magnetic resonance spectrum in DMSO-d6 of Compound 1 crystalline Form A hydrate.

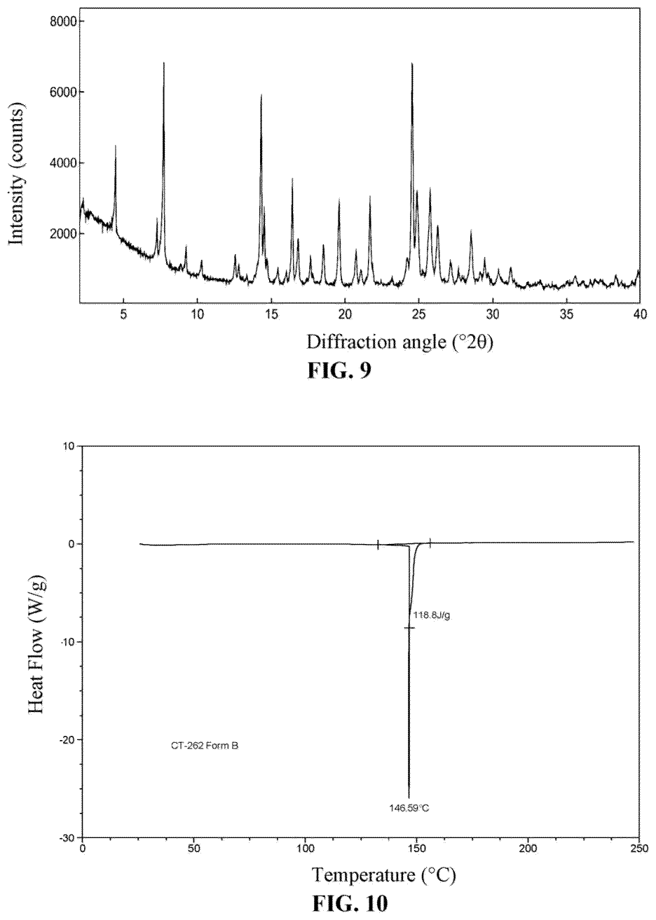

FIG. 9 shows a powder X-ray diffraction pattern diffractogram of Compound 1 crystalline Form B DMF solvate.

FIG. 10 shows a differential scanning calorimetry analysis curve of Compound 1 crystalline Form B DMF solvate.

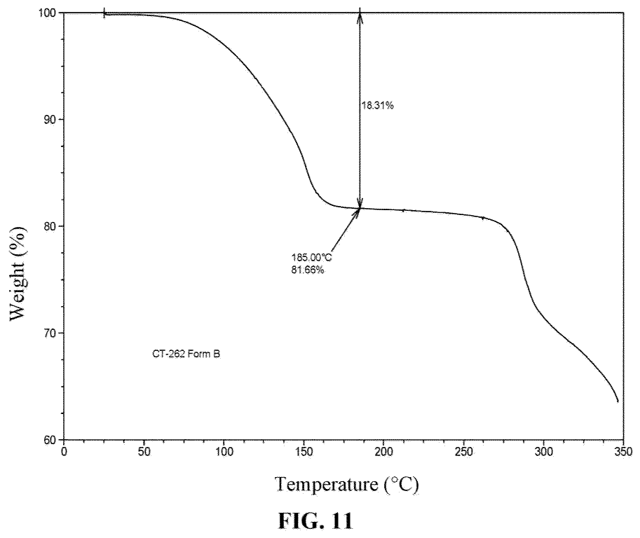

FIG. 11 shows a thermogravimetric analysis curve of Compound 1 crystalline Form B DMF solvate.

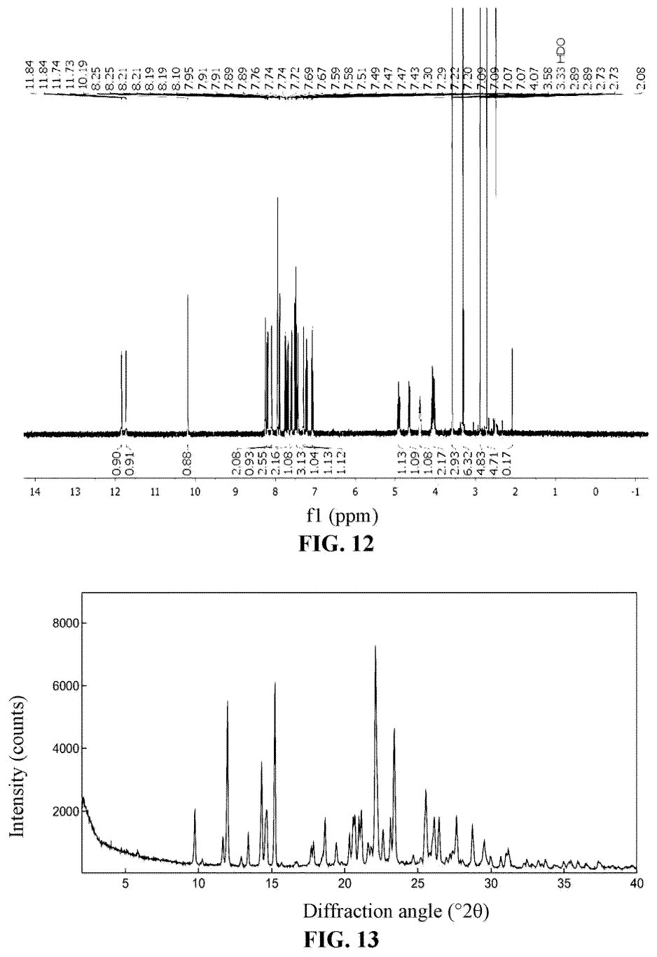

FIG. 12 shows a 1H nuclear magnetic resonance spectrum in DMSO-d6 of Compound 1 crystalline Form B DMF solvate.

FIG. 13 shows a powder X-ray diffraction pattern diffractogram of Compound 1 crystalline Form C anhydrate.

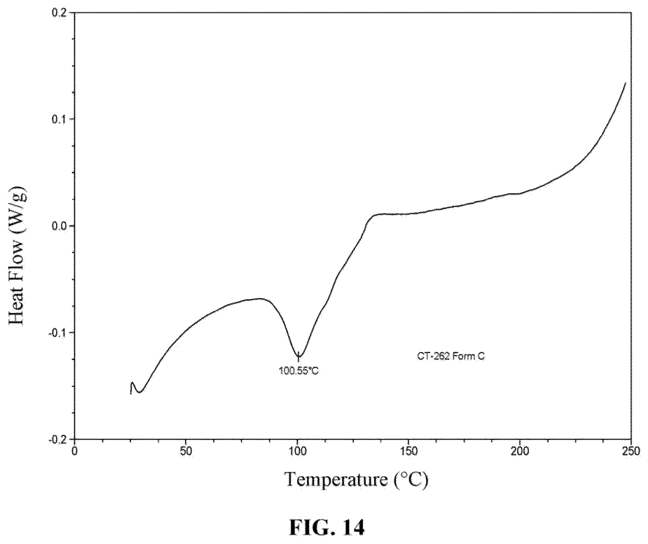

FIG. 14 shows a differential scanning calorimetry curve of Compound 1 crystalline Form C anhydrate.

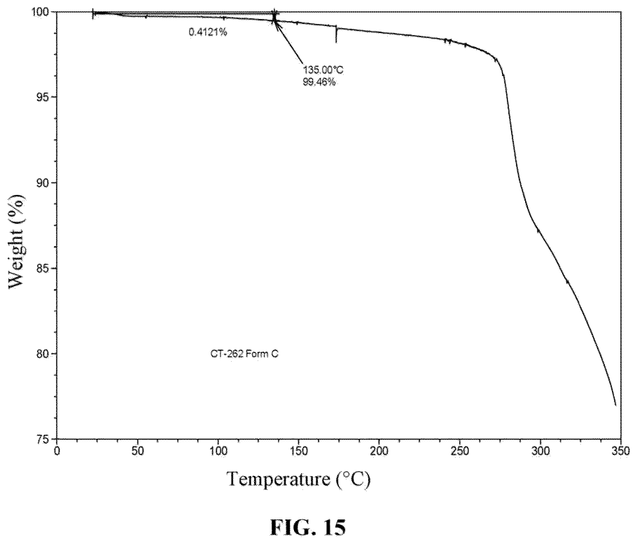

FIG. 15 shows a thermogravimetric analysis curve of Compound 1 crystalline Form C anhydrate.

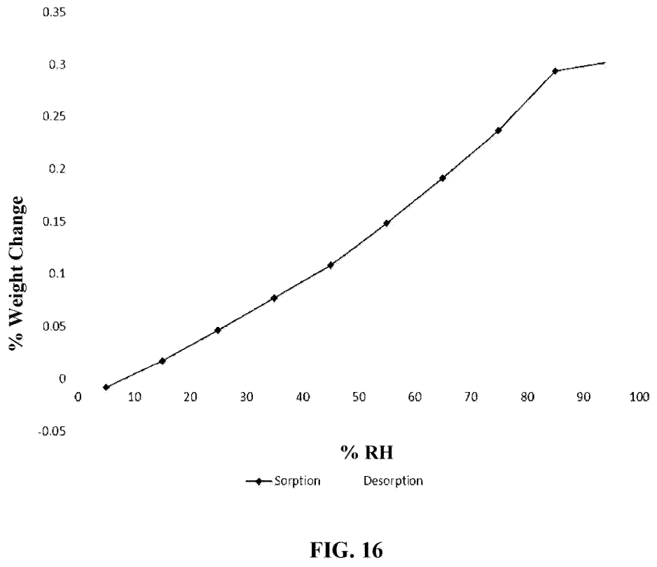

FIG. 16 shows a dynamic vapor sorption analysis of Compound 1 crystalline Form C anhydrate.

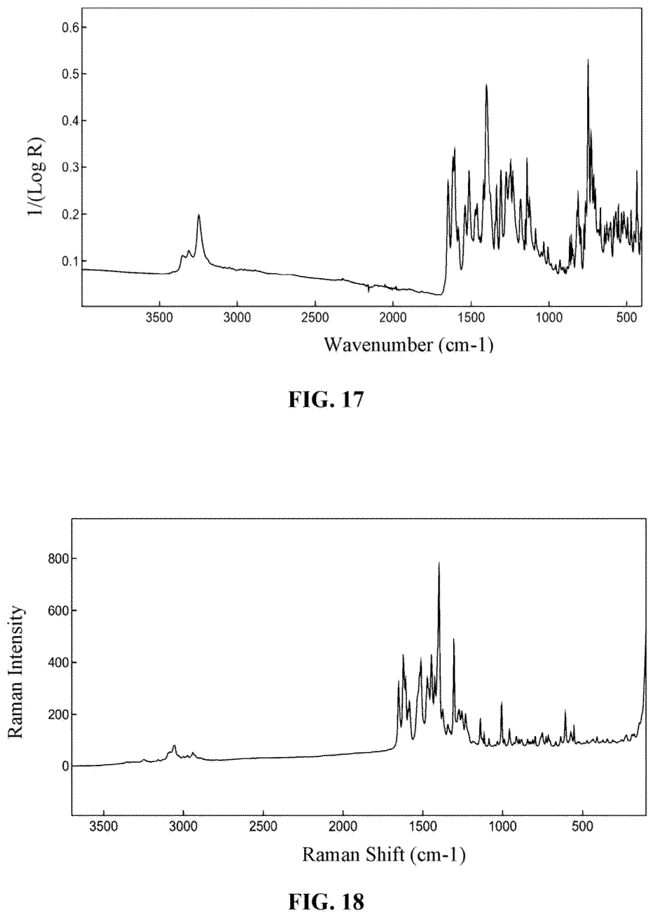

FIG. 17 shows an infrared spectrum of Compound 1 crystalline Form C anhydrate.

FIG. 18 shows a Raman spectrum of Compound 1 crystalline Form C anhydrate.

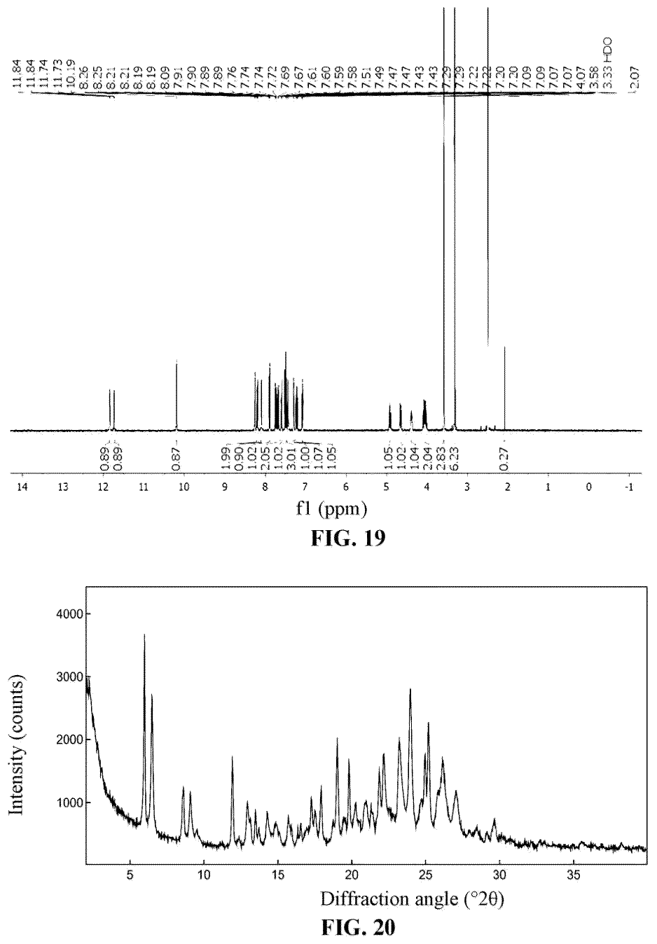

FIG. 19 shows a DMSO-d6 ¹H nuclear magnetic resonance spectrum of Compound 1 crystalline Form C anhydrate.

FIG. 20 shows a powder X-ray diffraction pattern diffractogram of Compound 1 crystalline Form E DMSO solvate.

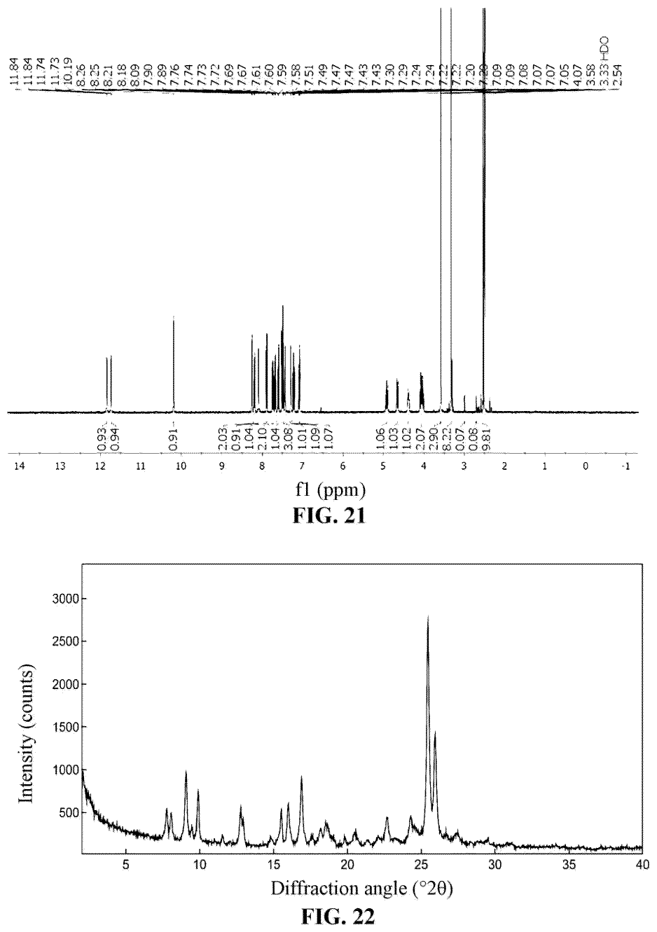

FIG. 21 shows a DMSO-d6 ¹H nuclear magnetic resonance spectrum of Compound 1 crystalline Form E DMSO solvate.

FIG. 22 shows a powder X-ray diffraction pattern diffractogram of Compound 1 crystalline Form F DMA solvate.

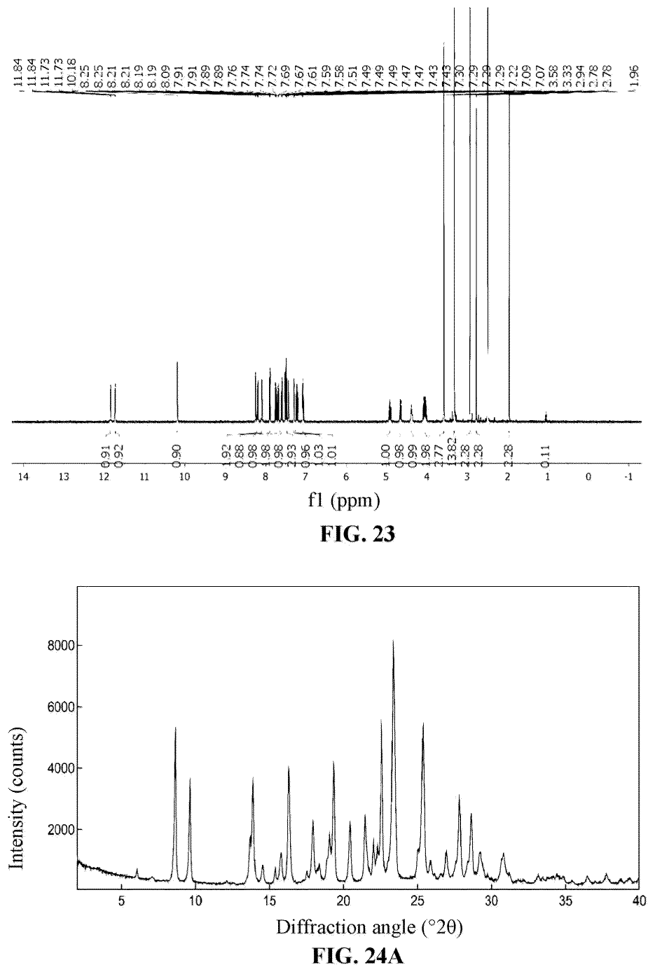

FIG. 23 shows a DMSO-d6 ¹H nuclear magnetic resonance spectrum of Compound 1 crystalline Form F DMA solvate.

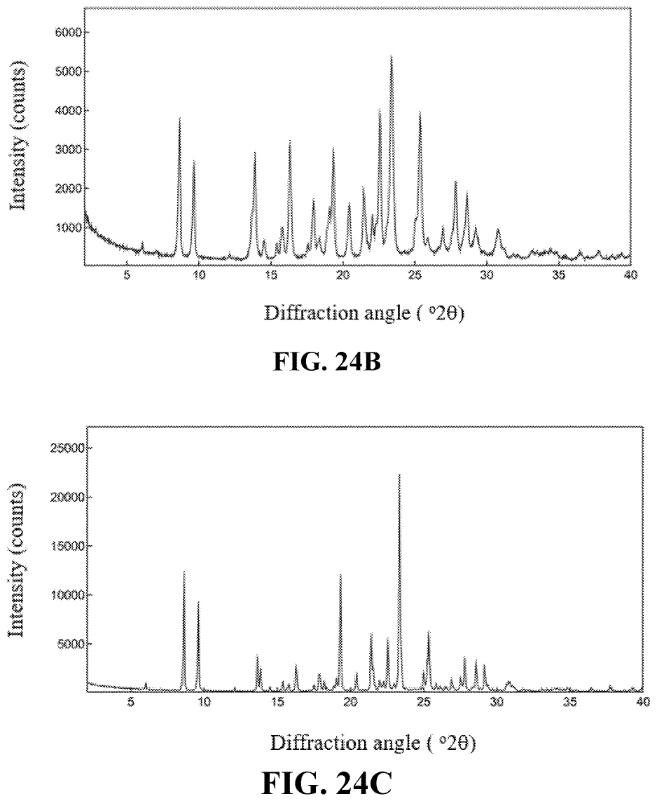

FIGS. 24A-24C show X-ray diffraction pattern diffractograms of Compound 1 crystalline Form A hydrate. The X-ray diffraction pattern diffractogram of FIG. 24A is from a 30 mg sample, slurried in methanol for two days at 40° C. FIG. 24B is from a 30 mg sample, slurried in methanol and water (about 95:about 5) for two days at 40° C. and subsequently allowed to air dry. The X-ray diffraction pattern diffractogram of FIG. 24C is from a 250 mg sample, slurried in methanol and water (about 95:about 5) for two days at 40° C. and subsequently allowed to air dry.

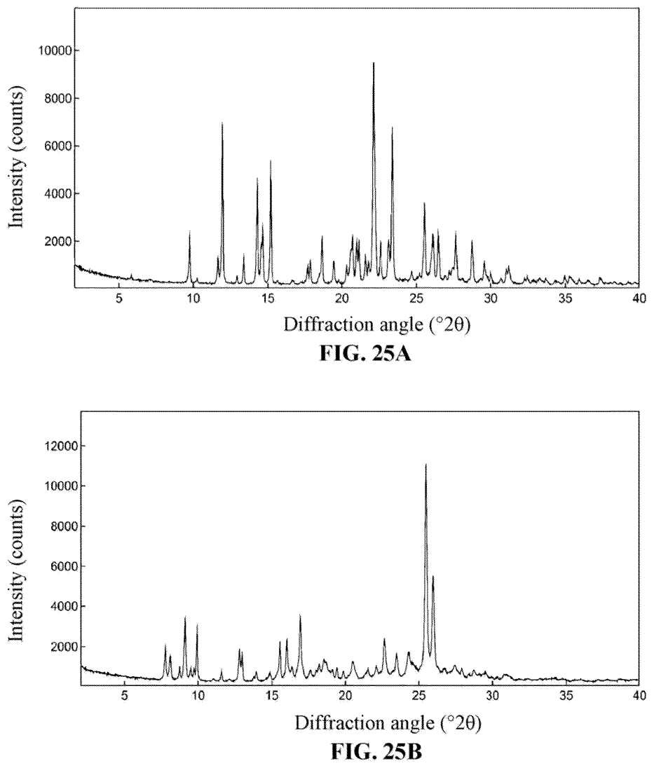

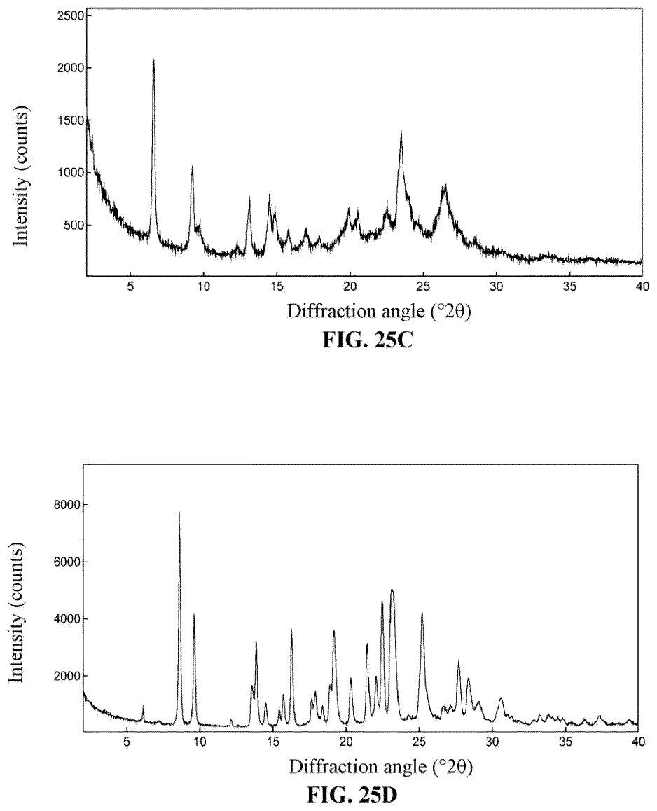

FIGS. 25A-25D show X-ray diffraction pattern diffractograms of various Compound 1 crystalline Forms. FIG. 25A shows a diffraction pattern diffractogram of crystalline Form C anhydrate. FIG. 25B shows a diffraction pattern diffractogram of a mixture of crystalline Form F DMA solvate and crystalline Form A hydrate. FIG. 25C shows a diffraction pattern diffractogram of crystalline Form E DMSO solvate. FIG. 25D shows a diffraction pattern diffractogram of crystalline Form A hydrate.

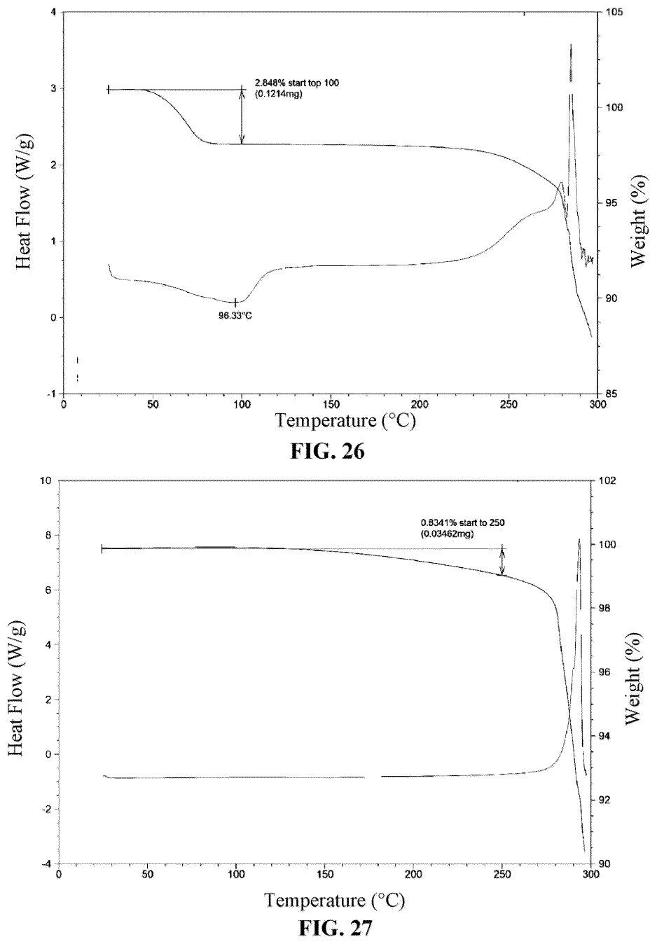

FIG. 26 shows a differential scanning calorimetry curve and a TG curve of Compound 1 crystalline Form A hydrate as prepared in Example 7.

FIG. 27 shows the differential scanning calorimetry curve and a TG curve of Compound 1 crystalline Form C anhydrate as prepared in Example 7.

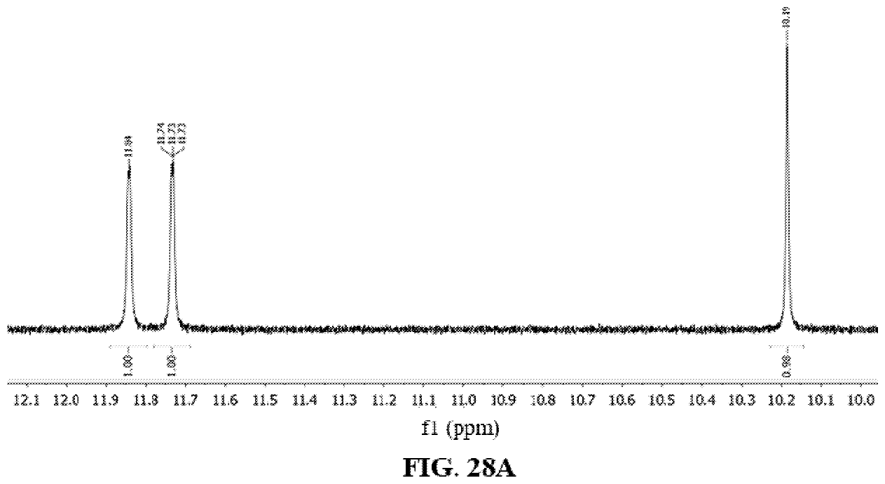

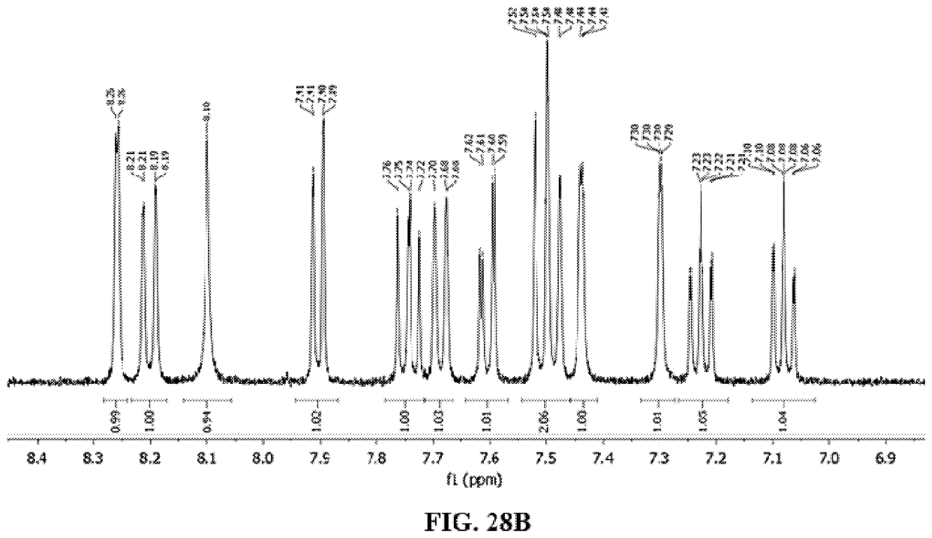

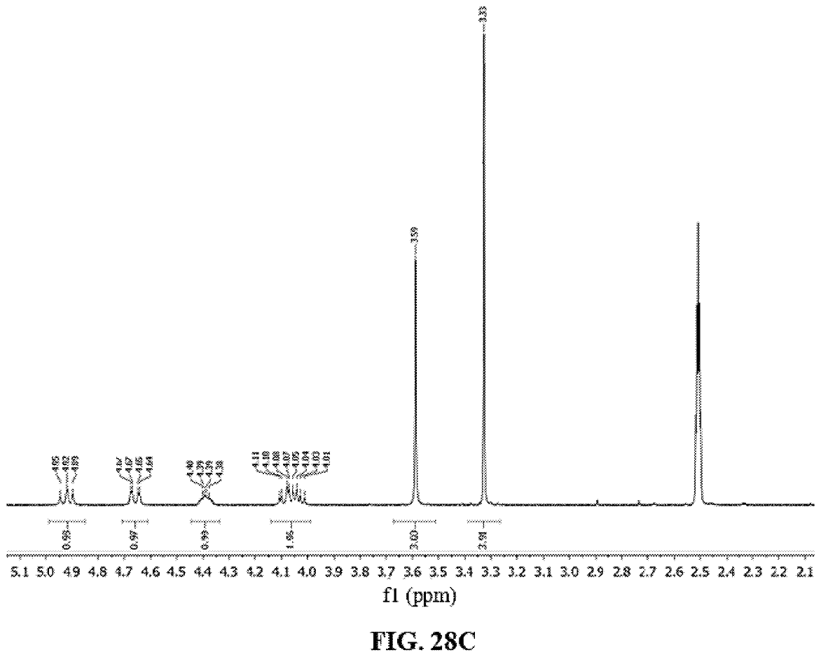

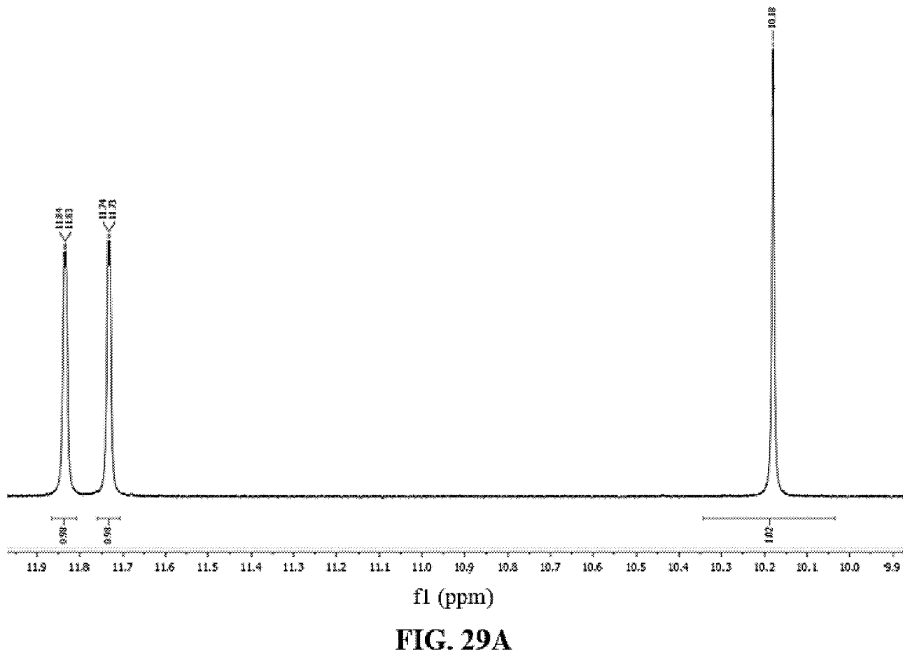

FIGS. 28A-28C show a DMSO-d6 ¹H nuclear magnetic resonance spectrum of Compound 1 crystalline Form A hydrate.

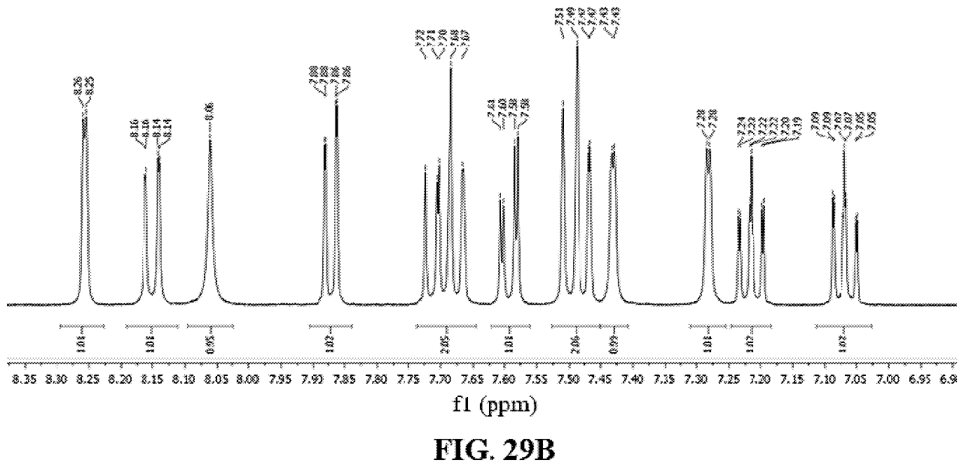

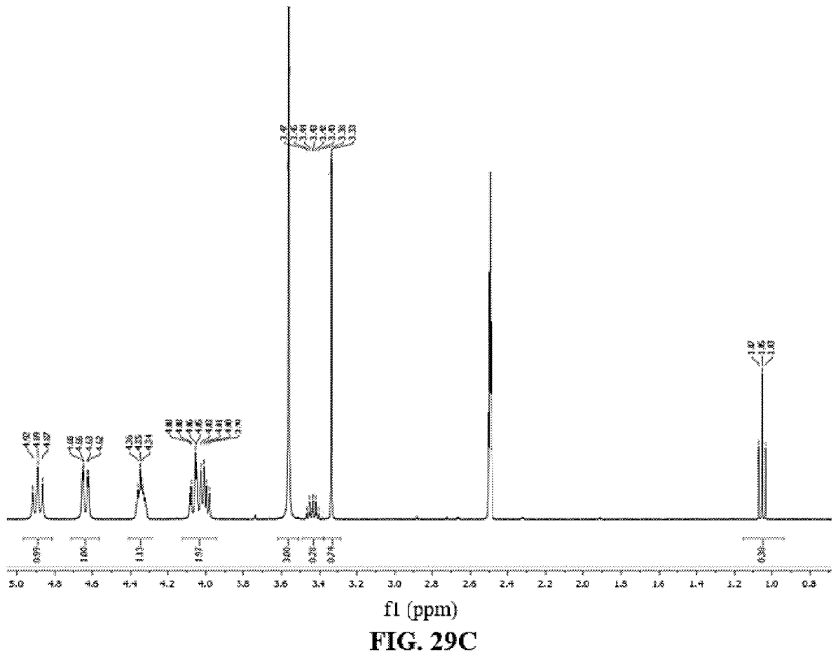

FIGS. 29A-29C show a DMSO-d6 ¹H nuclear magnetic resonance spectrum of Compound 1 crystalline Form C anhydrate.

### DETAILED DESCRIPTION

Within the scope of the present disclosure, it has been found that Compound 1 may take on various polymorphous crystal forms.

The XRPD spectra are indicative of 5 polymorphs observed, with crystalline Form A hydrate being that of a variable hydrate (water content of about 2.6%) based on the TG analysis, and crystalline Form C anhydrate being a non-solvated polymorph.

The present disclosure relates to novel crystalline polymorphic forms of Compound 1, and to processes for their preparation. Such polymorphic forms may be a component of a pharmaceutical composition and may be used to treat cancer.

In some embodiments, the disclosure provides Compound 1 crystalline Form A hydrate. In some embodiments, the crystalline Form A hydrate has an X-ray powder diffraction pattern substantially as shown in FIG. 2.

In some embodiments, the crystalline Form A hydrate is further characterized by an X-ray powder diffraction pattern comprising a peak at each of diffraction angles (2θ) 6.1±0.2°, 8.7±0.2°, 9.7±0.2°, 13.7±0.2°, 13.9±0.2°, 19.4±0.2°, 23.4±0.2, and 25.4±0.2°.

In some embodiments, the crystalline Form A hydrate is further characterized by an X-ray powder diffraction pattern comprising a peak at each of diffraction angles (2θ) 6.1±0.2°, 8.7±0.2°, 9.7±0.2°, 13.7±0.2°, and 13.9±0.2°.

In some embodiments, the crystalline Form A hydrate is further characterized by an X-ray powder diffraction pattern comprising a peak at each of diffraction angles (2θ) 8.7±0.2°, 9.7±0.2°, and 13.9±0.2°.

In some embodiments, the crystalline Form A hydrate has a differential scanning calorimetry thermogram (DSC) comprising an endothermic peak at about 78.0° C.

In some embodiments, the crystalline Form A hydrate has a differential scanning calorimetry thermogram (DSC) comprising a melting transition at about 279-281° C.

In some embodiments, the crystalline Form A hydrate has a differential scanning calorimetry thermogram (DSC) substantially as shown in FIG. 3.

In some embodiments, the crystalline Form A hydrate has a thermogravimetric analysis (TGA) substantially as shown in FIG. 4.

In some embodiments, the crystalline Form A hydrate is further characterized by an IR spectrum substantially depicted in FIG. 6.

In some embodiments, the crystalline Form A hydrate is further characterized by an IR spectrum having an absorption peak at each of 1426±2 cm⁻¹, 1547±2 cm⁻¹, and 1629±2 cm⁻¹.

In some embodiments, the crystalline Form A hydrate is further characterized by a Raman spectrum substantially depicted in FIG. 7.

In some embodiments, the crystalline Form A hydrate is further characterized by a Raman spectrum having a Raman shift at each of 1407±2 cm⁻¹, 1443±2 cm⁻¹, 1540±2 cm⁻¹, 1578±2 cm 1, and 1628±2 cm⁻¹.

In some embodiments, the crystalline Form A hydrate is further characterized by a Raman spectrum having a Raman shift at each of 1407±2 cm⁻¹, 1540±2 cm⁻¹, and 1578±2 cm⁻¹.

In some embodiments, the crystalline Form A hydrate is further characterized by a dynamic vapor sorption profile substantially as shown in FIG. 5.

In some embodiments, the crystalline Form A hydrate has a solution ¹H NMR spectrum profile in DMSO-d6 substantially as shown in FIG. 8.

In some embodiments, the crystalline Form A hydrate has a solution ¹H NMR spectrum DMSO-d6 comprising one or more peaks at about 3.58 ppm, 4.05 ppm, 4.38 ppm, 4.65 ppm, 4.91 ppm, 7.07 ppm, 7.22 ppm, 7.29 ppm, 7.43 ppm, 7.48 ppm, 7.60 ppm, 7.68 ppm, 7.74 ppm, 7.90 ppm, 8.09 ppm, 8.20 ppm, 8.25 ppm, 10.19 ppm, 11.73 ppm, and 11.84 ppm.

In some embodiments, the crystalline Form A hydrate has a water content of about 2.4 wt % of the crystalline Form A hydrate by Karl Fisher titration.

In some embodiments, the crystalline Form A hydrate is a nonstoichiometric hydrate.

In some embodiments, the disclosure provides a composition comprising Compound 1 crystalline Form A hydrate.

In some embodiments, the composition is a composition in which an effective amount of the crystalline Form A hydrate is dissolved.

In some embodiments, the compositions provided herein comprise a pharmaceutically acceptable carrier or diluent.

In some embodiments, the compositions are substantially free of Compound 1 crystalline Form C anhydrate.

In some embodiments, the compositions are compositions in which the crystalline Form A hydrate is dissolved, and the amount of the dissolved crystalline Form A hydrate is at least about 50% by weight of the composition.

In some embodiments, the compositions are compositions in which the crystalline Form A hydrate is dissolved, and the amount of the dissolved crystalline Form A hydrate is at least about 5% by weight of the composition.

In some embodiments, the compositions are compositions in which the crystalline Form A hydrate is dissolved, and the amount of the dissolved crystalline Form A hydrate is at least about 1% by weight of the composition.

In some embodiments, the crystalline Form A hydrate is a nonstoichiometric hydrate. The disclosure further provides a method of making the crystalline Form A hydrate, wherein the hydrate is a nonstoichiometric hydrate. The method of making the crystalline Form A hydrate comprises admixing Compound 1 and about 95:about 5 methanol:water (v/v) to obtain a slurry, and stirring the slurry at about 40° C. for about two days, where the slurry comprises the crystalline Form A hydrate.

In some embodiments, the method for making the crystalline Form A hydrate further comprises separating the crystalline Form A hydrate from the slurry and drying the separated crystalline Form A hydrate.

The disclosure further provides Compound 1 crystalline Form B DMF solvate.

In some embodiments, the crystalline Form B DMF solvate has an X-ray powder diffraction pattern substantially as shown in FIG. 9.

In some embodiments, the crystalline Form B DMF solvate is further characterized by an X-ray powder diffraction pattern comprising a peak at each of diffraction angles (2θ) 4.5±0.2°, 7.3±0.2°, 7.7±0.2°, 9.2±0.2°, and 10.3±0.2°.

In some embodiments, the crystalline Form B DMF solvate is further characterized by an X-ray powder diffraction pattern comprising a peak at each of diffraction angles (2θ) 4.5±0.2°, 7.3±0.2°, and 10.3±0.2°.

In some embodiments, the crystalline Form B DMF solvate has a differential scanning calorimetry thermogram (DSC) curve comprising an endothermic peak at about 147.0° C.

In some embodiments, the crystalline Form B DMF solvate has a differential scanning calorimetry thermogram (DSC) curve substantially as shown in FIG. 10.

In some embodiments, the crystalline Form B DMF solvate has a thermogravimetric analysis (TGA) curve substantially as shown in FIG. 11.

In some embodiments, the crystalline Form B DMF solvate has a solution ¹H NMR spectrum profile in DMSO-d6 substantially as shown in FIG. 12.

In some embodiments, the crystalline Form B DMF solvate has a solution ¹H NMR spectrum in DMSO-d6 comprising one or more peaks at about 3.58 ppm, 4.05 ppm, 4.38 ppm, 4.65 ppm, 4.91 ppm, 7.07 ppm, 7.22 ppm, 7.29 ppm, 7.43 ppm, 7.48 ppm, 7.60 ppm, 7.68 ppm, 7.74 ppm, 7.90 ppm, 7.95 ppm, 8.10 ppm, 8.20 ppm, 8.25 ppm, 10.19 ppm, 11.73 ppm, and 11.84 ppm.

In some embodiments, the crystalline Form B is a DMF solvate.

In some embodiments, the disclosure comprises a composition comprising the crystalline Form B DMF solvate.

In some embodiments, the compositions are compositions in which the crystalline Form B DMF solvate is dissolved.

In some embodiments, the compositions comprise a pharmaceutically acceptable carrier or diluent.

In some embodiments, the compositions are compositions in which the crystalline Form B DMF solvate is dissolved, and the dissolved crystalline Form B DMF solvate is at least about 50% by weight of the composition.

In some embodiments, the compositions are compositions in which the crystalline Form B DMF solvate is dissolved, and the dissolved crystalline Form B DMF solvate is at least about 5% by weight of the composition.

In some embodiments, the compositions are compositions in which the crystalline Form B DMF solvate is dissolved, and the dissolved crystalline Form B DMF solvate is at least about 1% by weight of the composition.

The disclosure further provides methods of making Compound 1 crystalline Form B DMF solvate, comprising admixing DMF with crystalline Form A hydrate to form a solution, and stirring the solution to obtain a slurry, where the slurry comprises the crystalline Form B DMF solvate.

The disclosure further provides methods of making Compound 1 crystalline Form B DMF solvate. The crystalline Form B DMF solvate may be made by admixing Form A hydrate and DMF to obtain a solution, and stirring the solution to obtain a slurry, wherein the slurry comprises the crystalline Form B DMF solvate.

The methods of making crystalline Form B DMF solvate may further comprise separating the crystalline Form B DMF solvate from the slurry and drying the separated crystalline Form B DMF solvate.

The disclosure further provides Compound 1 crystalline Form C anhydrate.

In some embodiments, the crystalline Form C anhydrate has an X-ray powder diffraction pattern substantially as shown in FIG. 13.

In some embodiments, the crystalline Form C anhydrate is further characterized by an X-ray powder diffraction pattern comprising a peak at each of diffraction angles (2θ) 9.8±0.2°, 11.7±0.2°, 12.0±0.2°, 13.4±0.2°, 15.2±0.2°.

In some embodiments, the crystalline Form C anhydrate is further characterized by an X-ray powder diffraction pattern comprising a peak at each of diffraction angles (2θ) 9.8±0.2°, 11.7±0.2°, and 15.2±0.2°.

In some embodiments, the crystalline Form C anhydrate has a differential scanning calorimetry thermogram (DSC) curve substantially as shown in FIG. 14.

In some embodiments, the crystalline Form C anhydrate has a differential scanning calorimetry thermogram (DSC) curve comprising an endothermic peak at about 100.55° C. In some embodiments, the crystalline Form C anhydrate has a thermogravimetric analysis (TGA) curve substantially as shown in FIG. 15.

In some embodiments, the crystalline Form C anhydrate is further characterized by an IR spectrum substantially depicted in FIG. 17.

In some embodiments, the crystalline Form C anhydrate is further characterized by an IR spectrum having an absorption peak at each of 1402±2 cm⁻¹, 1421±2 cm⁻¹, 1539±2 cm⁻¹, and 1616±2 cm⁻¹.

In some embodiments, the crystalline Form C anhydrate is further characterized by a Raman spectrum substantially depicted in FIG. 18.

In some embodiments, the crystalline Form C anhydrate is further characterized by a Raman spectrum having a Raman shift at each of 1399±2 cm⁻¹, 1584±2 cm⁻¹, and 1623±2 cm⁻¹.

In some embodiments, the crystalline Form C anhydrate is further characterized by a dynamic vapor sorption profile substantially as shown in FIG. 16.

In some embodiments, the crystalline Form C anhydrate has a solution ¹H NMR spectrum profile in DMSO-d6 substantially as shown in FIG. 19.

In some embodiments, the crystalline Form C anhydrate has a solution ¹H NMR spectrum DMSO-d6 comprising one or more peaks at about 3.58 ppm, 4.05 ppm, 4.38 ppm, 4.65 ppm, 4.91 ppm, 7.07 ppm, 7.22 ppm, 7.29 ppm, 7.43 ppm, 7.49 ppm, 7.60 ppm, 7.68 ppm, 7.74 ppm, 7.90 ppm, 8.09 ppm, 8.20 ppm, 8.25 ppm, 10.19 ppm, 11.73 ppm, and 11.84 ppm.

In some embodiments, the disclosure provides a composition comprising Compound 1 crystalline Form C anhydrate.

In some embodiments, the compositions are compositions in which the crystalline Form C anhydrate is dissolved.

In some embodiments, the compositions comprise a pharmaceutically acceptable carrier or diluent.

In some embodiments, the compositions are substantially free of Compound 1 crystalline Form A hydrate.

In some embodiments, the compositions are compositions in which the crystalline Form C anhydrate is dissolved, and the dissolved crystalline Form C anhydrate is at least about 50% by weight of the composition.

In some embodiments, the compositions are compositions in which the crystalline Form C anhydrate is dissolved, and the dissolved crystalline Form C anhydrate is at least about 5% by weight of the composition.

In some embodiments, the compositions are compositions in which the crystalline Form C anhydrate is dissolved, and the dissolved crystalline Form C anhydrate is at least about 1% by weight of the composition.

The disclosure further provides methods of making Compound 1 crystalline Form C anhydrate. The crystalline Form C anhydrate may be made by admixing crystalline Form A hydrate and acetonitrile or ethanol to obtain a slurry, and stirring the slurry, wherein the slurry comprises the crystalline Form C anhydrate.

The disclosure further provides a method of making Compound 1 crystalline Form C anhydrate. comprising admixing acetonitrile and crystalline Form A hydrate to form a slurry, and stirring the solution at about 40° C. for about one week, wherein the slurry comprises the crystalline Form C anhydrate.

In some embodiments, the methods of making the crystalline Form C anhydrate further comprise separating the crystalline Form C anhydrate from the slurry and drying the separated crystalline Form C anhydrate.

The disclosure further provides methods of making Compound 1 crystalline Form C anhydrate, comprising admixing absolute ethanol and crystalline Form A hydrate to form a slurry, and stirring the slurry at about 40° C. for about two days, where the slurry comprises the crystalline Form C anhydrate.

The disclosure further provides methods of making Compound 1 crystalline Form C anhydrate, comprising admixing absolute ethanol and crystalline Form A hydrate to form a slurry, and stirring the slurry at about 50° C. for about one hour, where the slurry comprises the crystalline Form C anhydrate.

In some embodiments, the methods of making crystalline Form C anhydrate further comprise separating the crystalline Form C anhydrate from the slurry and drying the separated crystalline Form C anhydrate.

The disclosure further provides Compound 1 crystalline Form E DMSO solvate.

In some embodiments, the crystalline Form E DMSO solvate has an X-ray powder diffraction pattern substantially as shown in FIG. 20.

In some embodiments, the crystalline Form E DMSO solvate is further characterized by an X-ray powder diffraction pattern comprising a peak at each of diffraction angles (2θ) 6.0±0.2°, 6.5±0.2°, 8.6±0.2°, 9.1±0.2°, 11.9±0.2°.

In some embodiments, the crystalline Form E DMSO solvate is further characterized by an X-ray powder diffraction pattern comprising a peak at each of diffraction angles (2θ) 6.0±0.2°, 6.5±0.2°, and 8.6±0.2°.

In some embodiments, the crystalline Form E DMSO solvate has a solution ¹H NMR spectrum profile in DMSO-d6 substantially as shown in FIG. 21.

In some embodiments, the crystalline Form E has a solution ¹H NMR spectrum in DMSO-d6 comprising one or more peaks at about 2.53 ppm, 3.58 ppm, 4.05 ppm, 4.38 ppm, 4.65 ppm, 4.91 ppm, 7.07 ppm, 7.22 ppm, 7.29 ppm, 7.43 ppm, 7.48 ppm, 7.60 ppm, 7.68 ppm, 7.74 ppm, 7.90 ppm, 8.09 ppm, 8.20 ppm, 8.25 ppm, 10.19 ppm, 11.73 ppm, and 11.84 ppm.

In some embodiments, the crystalline Form E is a DMSO solvate.

The disclosure further provides methods of making Compound 1 crystalline Form E DMSO solvate. The crystalline Form E DMSO solvate may be made by admixing crystalline Form A hydrate and DMSO to obtain a solution, followed by adding to the solution water dropwise to obtain a slurry and stirring the slurry, wherein the slurry comprises the crystalline Form E DMSO solvate

The disclosure further provides methods of making Compound 1 crystalline Form E DMSO solvate, the method comprising: admixing DMSO and crystalline Form A hydrate to form a solution, adding to the solution water dropwise to form a slurry, and stirring the slurry for about one day, wherein the slurry comprises the crystalline Form E DMSO solvate.

In some embodiments, the methods further comprise separating the crystalline Form E DMSO solvate from the slurry and drying the separated crystalline Form E DMSO solvate.

In some embodiments, the disclosure provides a composition comprising the crystalline Form E DMSO solvate.

In some embodiments, the compositions are compositions in which the crystalline Form E DMSO solvate is dissolved.

In some embodiments, the compositions comprise a pharmaceutically acceptable carrier or diluent.

In some embodiments, the compositions are compositions in which the crystalline Form E DMSO solvate dissolved, and the dissolved crystalline Form E DMSO solvate is at least about 50% by weight of the composition.

In some embodiments, the compositions are compositions in which the crystalline Form E DMSO solvate dissolved, and the dissolved crystalline Form E DMSO solvate is at least about 5% by weight of the composition.

In some embodiments, the compositions are compositions in which the crystalline Form E DMSO solvate is dissolved, and the dissolved crystalline Form E DMSO solvate is at least about 1% by weight of the composition.

The disclosure further provides Compound 1 crystalline Form F DMA solvate.

In some embodiments, the crystalline Form F DMA solvate has an X-ray powder diffraction pattern substantially as shown in FIG. 22.

In some embodiments, the crystalline Form F DMA solvate is further characterized by an X-ray powder diffraction pattern comprising a peak at each of diffraction angles (2θ) 7.8±0.2°, 8.1±0.2°, 9.1±0.2°, 9.5±0.2°, and 9.9±0.2°.

In some embodiments, the crystalline Form F DMA solvate is further characterized by an X-ray powder diffraction pattern comprising a peak at each of diffraction angles (2θ) 8.1±0.2°, 9.1±0.2°, and 9.9±0.2.

In some embodiments, the crystalline Form F DMA solvate is a solution ¹H NMR spectrum profile in DMSO-d6 substantially as shown in FIG. 23.

In some embodiments, the crystalline Form F DMA solvate is a solution ¹H NMR spectrum in DMSO-de comprising one or more peaks at about 1.95 ppm, 2.78 ppm, 2.94 ppm, 3.58 ppm, 4.05 ppm, 4.38 ppm, 4.65 ppm, 4.91 ppm, 7.07 ppm, 7.22 ppm, 7.29 ppm, 7.43 ppm, 7.48 ppm, 7.60 ppm, 7.68 ppm, 7.74 ppm, 7.90 ppm, 8.09 ppm, 8.20 ppm, 8.25 ppm, 10.19 ppm, 11.73 ppm, and 11.84 ppm.

In some embodiments, the crystalline Form F DMA solvate is a mono-DMA solvate.

The disclosure further provides compositions comprising the crystalline Form F DMA solvate.

In some embodiments, the compositions are compositions in which the crystalline Form F DMA solvate is dissolved.

The disclosure further provides methods for treating cancer in a mammal in need thereof, comprising the step of administering to the mammal an effective amount of any of Compound 1 crystalline Forms A-F. In some embodiments, the methods comprise administering to the mammal a composition in which any of Forms A-F is dissolved.

In some embodiments, the compositions comprise a pharmaceutically acceptable carrier or diluent.

In some embodiments, the compositions are compositions in which the crystalline Form F DMA solvate is dissolved, and the dissolved crystalline Form F DMA solvate is at least about 50% by weight of the composition.

In some embodiments, the compositions are compositions in which the crystalline Form F DMA solvate is dissolved, and the dissolved crystalline Form F DMA solvate is at least about 5% by weight of the composition.

In some embodiments, the compositions are compositions in which the crystalline Form F DMA solvate is dissolved, and the dissolved crystalline Form F DMA solvate is at least about 1% by weight crystalline by weight of the composition.

The disclosure further provides methods of making Compound 1 crystalline Form F DMA solvate. The crystalline Form F DMA solvate may be made by admixing (i) a mixture of crystalline Form A hydrate and Form C anhydrate and (ii) DMA and H2O to obtain a slurry and stirring the slurry, wherein the slurry comprises the crystalline Form F DMA solvate.

The disclosure further provides methods of making Compound 1 crystalline Form F DMA solvate, the method comprising: admixing about 70:about 30 DMA:H₂O and a mixture of crystalline Form A hydrate and crystalline Form C anhydrate to form a slurry, and stirring the slurry for about one week, wherein the slurry comprises the crystalline Form F DMA solvate.

In some embodiments, the DMA is “dry” DMA. As used herein, “dry” mean that the DMA comprises no more than about 0.01% % water by weight of the DMA.

In some embodiments, the method of making crystalline Form F DMA solvate further comprises separating the crystalline Form F DMA solvate from the slurry and drying the separated crystalline Form F DMA solvate.

The disclosure further provides a Compound 1 crystalline Form A nonstoichiometric hydrate, further characterized by at a temperature of 50° C. in absolute ethanol for about one hour, the Compound 1 crystalline Form A hydrate converts to the crystalline Form C anhydrate.

Further provided are methods for making Compound 1 crystalline Form C anhydrate, comprising heating a solution of Compound 1 crystalline Form A hydrate in absolute ethanol at a temperature of about 50° C. for about one hour to provide Compound 1 crystalline Form C anhydrate.

The disclosure further provides Compound 1 crystalline Form A nonstoichiometric hydrate, further characterized by at a temperature of about 40° C. in absolute ethanol for about two days, the Compound 1 crystalline Form A hydrate converts to the crystalline Form C anhydrate.

Further provided are methods for making Compound 1 crystalline Form C anhydrate, comprising heating a solution of Compound 1 crystalline Form A hydrate in absolute ethanol at a temperature of about 40° C. for about two days to provide Compound 1 crystalline Form C anhydrate.

Further provided are methods for making Compound 1 crystalline Form C anhydrate, comprising heating a solution of Compound 1 crystalline Form A hydrate in absolute ethanol at a temperature of about 40° C. for about two days to provide Compound 1 crystalline Form C anhydrate.

The disclosure further provides Compound 1 crystalline Form A hydrate that is substantially free of Compound 1 crystalline Form C anhydrate.

The disclosure further provides Compound 1 crystalline Form C of anhydrate of Compound 1 that is substantially free of Compound 1 crystalline Form A hydrate.

The disclosure further provides methods for treating cancer in a mammal in need thereof, comprising the step of administering to the mammal an effective amount of a Compound 1 crystalline form, wherein the crystalline form is crystalline Form A hydrate, crystalline Form B DMF solvate, crystalline Form C anhydrate, crystalline Form E DMSO solvate, crystalline Form F DMA solvate or a mixture of the foregoing.

The disclosure further provides methods for treating cancer in a mammal in need thereof, comprising the step of administering to the mammal an effective amount of a composition in which Compound 1 crystalline Form A hydrate, Compound 1 crystalline Form B DMF solvate, Compound 1 crystalline Form C anhydrate, Compound 1 crystalline Form E DMSO solvate, or Compound 1 crystalline Form F DMA solvate, or a mixture of the foregoing, is dissolved.

Some embodiments comprise administering an effective amount of Compound 1 crystalline Form A hydrate or an effective amount of a composition in which Compound 1 crystalline Form A hydrate is dissolved.

In some embodiments, the crystalline Form A hydrate is a variable hydrate between hemihydrate and monohydrate. In some embodiments, the crystalline Form A is a hydrate comprising 0.5 to 0.8 molar equivalents of water per molar equivalent of crystalline Form A. In some embodiments, the crystalline Form A hydrate is a nonstoichiometric hydrate.

Some embodiments comprise administering an effective amount of Compound 1 crystalline Form A hydrate or an effective amount of a composition in which Compound 1 crystalline Form A hydrate is dissolved, wherein the Compound 1 crystalline Form A hydrate has an X-ray powder diffraction pattern substantially as shown in FIG. 2. In some embodiments, the crystalline Form A hydrate is further characterized by an X-ray powder diagram that has characteristic reflections at the following d values: 14.4886 Å, 10.1636 Å, 9.1368, Å, 6.4541 Å, and 6.3709 Å.

In some embodiments, the crystalline Form A hydrate is additionally characterized by in an X-ray powder diffraction pattern that may comprise a peak at each of diffraction angles (2θ): 6.1±0.2°, 8.7±0.2°, 9.7±0.2°, 13.7±0.2°, 13.9±0.2°, 19.4±0.2°, 23.4±0.2° and 25.4±0.2°.

In some embodiments, the crystalline Form A hydrate is additionally characterized by an X-ray powder diffraction pattern that may comprise a peak at each of diffraction angles (2θ): 6.1±0.2°, 8.7±0.2°, 9.7±0.2°, 13.7±0.2°, and 13.9±0.2°.

In some embodiments, the crystalline Form A hydrate is additionally characterized by an X-ray powder diffraction pattern that may comprise a peak at each of diffraction angles (2θ): 8.7±0.2°, 9.7±0.2°, and 13.9±0.2°.

Some embodiments comprise administering an effective amount of crystalline Form A hydrate or an effective amount of a composition in which Compound 1 crystalline Form A hydrate is dissolved, wherein the Compound 1 crystalline Form A hydrate has a differential scanning calorimetry thermogram (DSC) that may comprise an endothermic peak at about 78.0° C., and a melting transition at about 279-281° C. In some embodiments, the crystalline Form A hydrate is additionally characterized as having a differential scanning calorimetry thermogram (DSC) substantially as shown in FIG. 3. In some embodiments, the crystalline Form A hydrate is additionally characterized as having a thermogravimetric analysis (TGA) substantially as shown in FIG. 4.

Some embodiments comprise administering an effective amount of Compound 1 crystalline Form A hydrate or an effective amount of a composition in which Compound 1 crystalline Form A hydrate is dissolved, wherein the Compound 1 crystalline Form A hydrate has an IR spectrum having an absorption peak at each of 1426±2 cm⁻¹, 1547±2 cm⁻¹, and 1629±2 cm⁻¹.

Some embodiments comprise administering an effective amount of Compound 1 crystalline Form A hydrate characterized by a Raman spectrum having a Raman shift at each of 1407±2 cm⁻¹, 1443±2 cm⁻¹, 1540±2 cm⁻¹, 1578±2 cm⁻¹, and 1628±2 cm⁻¹.

Some embodiments comprise administering an effective amount of Compound 1 crystalline Form A hydrate or an effective amount of a composition in which Compound 1 crystalline Form A hydrate is dissolved, wherein the Compound 1 crystalline Form A hydrate has a solution 1H NMR spectrum in DMSO-d6 comprising one or more peaks at about 3.58 ppm, 4.05 ppm, 4.38 ppm, 4.65 ppm, 4.91 ppm, 7.07 ppm, 7.22 ppm, 7.29 ppm, 7.43 ppm, 7.48 ppm, 7.60 ppm, 7.68 ppm, 7.74 ppm, 7.90 ppm, 8.09 ppm, 8.20 ppm, 8.25 ppm, 10.19 ppm, 11.73 ppm, and 11.84 ppm.

Some embodiments comprise administering an effective amount of Compound 1 crystalline Form A hydrate or an effective amount of a composition in which Compound 1 crystalline Form A hydrate is dissolved, wherein the Compound 1 crystalline Form A hydrate has a water content of about 2.4 wt % of the crystalline Form A hydrate by Karl Fisher titration.

Some embodiments comprise administering an effective amount of a composition comprising the crystalline Form A hydrate or an effective amount of a composition in which Compound 1 crystalline Form A hydrate is dissolved. Said composition may comprise at least one pharmaceutically acceptable carrier or diluent.

Some embodiments comprise administering an effective amount of the composition, in which Compound 1 Form A hydrate is dissolved, wherein the dissolved Compound 1 Form A hydrate is about 0.1-75% % by weight of the composition inclusive, comprising embodiments where the dissolved Compound 1 Form A hydrate is about 1% by weight of the composition, 5% by weight crystalline of the composition, for example.

In some embodiments, provided herein are methods of making a Compound 1 crystalline Form A hydrate. The hydrate (which may be a non-stoichiometric hydrate) may be formed by admixing methanol and water and Compound 1 to form a slurry, and stirring the slurry, wherein the slurry comprises the crystalline Form A hydrate.

The disclosure further provides methods of making Compound 1 crystalline Form A nonstoichiometric hydrate, comprising admixing about 95:about 5 methanol:water (v/v) and Compound 1 to form a slurry and stirring the slurry at about 40° C. for about two days, wherein the slurry comprises crystalline Form A hydrate. In some embodiments, the crystalline Form A hydrate is separated from the slurry and dried or allowed to dry to obtain crystalline Form A hydrate.

Some embodiments comprise administering an effective amount of the crystalline Form A hydrate that is substantially free of the Compound 1 crystalline Form C anhydrate or administering an effective amount of a composition in which crystalline Form A hydrate that is substantially free of crystalline Form C anhydrate is dissolved. Crystalline Form C anhydrate of Compound 1 is described below.

In some embodiments, the composition comprises less than about 4% (w/w) of crystalline Form C anhydrate. In some embodiments, the compositions comprise crystalline Form A hydrate that is substantially free of crystalline Form C anhydrate, wherein the composition comprises less than about 2% (w/w) of crystalline Form C anhydrate.

The disclosure further provides methods for making a composition comprising Compound 1 crystalline Form A hydrate that is substantially free of Compound 1 crystalline Form C anhydrate.

In some embodiments, Compound 1 crystalline Form A nonstoichiometric hydrate is further characterized in that at a temperature of 50° C. in absolute ethanol for about one hour, the crystalline Form A hydrate converts to the Compound 1 crystalline Form C anhydrate.

In some embodiments, Compound 1 crystalline Form A nonstoichiometric hydrate is further characterized in that at a temperature of about 40° C. in absolute ethanol for about two days, the crystalline Form A hydrate converts to Compound 1 crystalline Form C anhydrate.

Further provided are methods for making Compound 1 crystalline Form C anhydrate, comprising heating a solution of Compound 1 crystalline Form A hydrate nonstoichiometric hydrate in absolute ethanol at a temperature of about 40° C. for about two days to provide Compound 1 crystalline Form C anhydrate.

Some embodiments comprise the Compound 1 crystalline Form B of a DMF solvate.

Some embodiments comprise the Compound 1 crystalline Form B DMF solvate having an X-ray powder diffraction pattern substantially as shown in FIG. 9. Compound 1 Crystalline Form B DMF solvate is further characterized by an X-ray powder diagram that has characteristic reflections at the following d values: 19.8119 Å, 12.1760 Å, 11.4515 Å, 9.5708 Å, and 8.5881 Å.

In some embodiments, Compound 1 crystalline Form B DMF solvate is characterized by an X-ray powder diffraction pattern that may comprise a peak at each of diffraction angles (2θ): 4.5±0.2°, 7.3±0.2°, 7.7±0.2°, 9.2±0.2°, and 10.3±0.2°.

In some embodiments, crystalline Form B DMF solvate has a differential scanning calorimetry thermogram (DSC) that may comprise an endothermic peak at about 147.0° C. as shown in FIG. 10.

In some embodiments, Compound 1 crystalline Form B DMF solvate is additionally characterized as having a differential scanning calorimetry thermogram (DSC) substantially as shown in FIG. 10.

In some embodiments, Compound 1 crystalline Form B DMF solvate is additionally characterized as having a thermogravimetric analysis (TGA) substantially as shown in FIG. 11.

In some embodiments, Compound 1 crystalline Form B DMF solvate is characterized by TGA analysis in that the crystalline Form B DMF solvate undergoes an 18.3% weight loss from start, to 185° C. as shown in FIG. 11.

Some embodiments comprise Compound 1 crystalline Form B DMF solvate having a solution ¹H NMR spectrum in DMSO-d6 comprising one or more peaks at about 3.58 ppm, 4.05 ppm, 4.38 ppm, 4.65 ppm, 4.91 ppm, 7.07 ppm, 7.22 ppm, 7.29 ppm, 7.43 ppm, 7.48 ppm, 7.60 ppm, 7.68 ppm, 7.74 ppm, 7.90 ppm, 7.95 ppm, 8.10 ppm, 8.20 ppm, 8.25 ppm, 10.19 ppm, 11.73 ppm, and 11.84 ppm. In some embodiments, crystalline Form B is a DMF solvate.

Some embodiments comprise a method of making crystalline Form B as a DMF solvate of Compound 1. The method of making crystalline Form B DMF solvate comprises admixing DMF and crystalline Form A hydrate to form a solution, stirring the solution to obtain a slurry, where the slurry comprises crystalline Form B DMF solvate. Additional embodiments of the method are where crystalline Form B DMF solvate is separated from the slurry and dried or allowed to dry to form crystalline Form B DMF solvate.

Some embodiments comprise one or more of the following features: the composition where the composition is about 1-75% % by weight crystalline Form B DMF solvate inclusive, comprising embodiments where the composition is about 1% by weight crystalline Form B DMF solvate, and about 5% by weight crystalline Form B DMF solvate, for example. Said composition may comprise at least one pharmaceutically acceptable carrier or diluent.

Some embodiments comprise the Compound 1 crystalline Form C.

Some embodiments comprise Compound 1 crystalline Form C anhydrate having an X-ray powder diffraction pattern substantially as shown in FIG. 13.

In some embodiments, Compound 1 crystalline Form C anhydrate is further characterized by an X-ray powder diagram that has characteristic reflections at the following d values: 9.0620 Å, 7.5893 Å, 7.3873 Å, 6.6075 Å, and 5.8212 Å.

In some embodiments, Compound 1 crystalline Form C anhydrate is further characterized by an X-ray powder diagram that has characteristic reflections at the following d values: 9.0620 Å, 7.5893 Å, and 5.8212 Å.

In some embodiments, Compound 1 crystalline Form C anhydrate is characterized by an x-ray powder diffraction pattern that may comprise a peak at each of diffraction angles (2θ): 9.8±0.2°, 11.7±0.2°, 12.0±0.2°, 13.4±0.2°, and 15.2±0.2°.

In some embodiments, Compound 1 crystalline Form C anhydrate is characterized by an x-ray powder diffraction pattern that may comprise a peak at each of diffraction angles (2θ): 9.8±0.2°, 11.7±0.2°, and 15.2±0.2°.

In some embodiments, Compound 1 crystalline Form C anhydrate is additionally characterized as having a differential scanning calorimetry thermogram (DSC) substantially as shown in FIG. 14.

In some embodiments, the Compound 1 crystalline Form C anhydrate is also characterized by having a differential scanning calorimetry thermogram (DSC) that may comprise an endothermic peak at about 100.55° C.

Some embodiments comprise Compound 1 crystalline Form C anhydrate having a thermogravimetric analysis (TGA) substantially as shown in FIG. 15. In some embodiments, crystalline Form C anhydrate is characterized by TGA analysis in that the crystalline Form C anhydrate undergoes about 0.4% weight loss from start, to about 135° C. as shown in FIG. 15.

In some embodiments, Compound 1 crystalline Form C anhydrate is also characterized by a dynamic vapor sorption profile substantially as shown in FIG. 16.

In some embodiments, Compound 1 crystalline Form C anhydrate is also characterized by an IR spectrum substantially depicted in FIG. 17. In some embodiments, crystalline Form C anhydrate is further characterized by an IR spectrum having an absorption peak at each of 1402±2 cm⁻¹, 1421±2 cm⁻¹, 1539±2 cm⁻¹, and 1616±2 cm⁻¹. In some embodiments, crystalline Form C anhydrate is also characterized by a Raman spectrum substantially depicted in FIG. 18. In some embodiments, crystalline Form C anhydrate is further characterized by a Raman spectrum having a Raman shift at each of 1399±2 cm⁻¹, 1584±2 cm⁻¹, and 1623±2 cm⁻¹.

Some embodiments comprise Compound 1 crystalline Form C anhydrate having a solution ¹H NMR spectrum in DMSO-d6 comprising one or more peaks at about 3.58 ppm, 4.05 ppm, 4.38 ppm, 4.65 ppm, 4.91 ppm, 7.07 ppm, 7.22 ppm, 7.29 ppm, 7.43 ppm, 7.49 ppm, 7.60 ppm, 7.68 ppm, 7.74 ppm, 7.90 ppm, 8.09 ppm, 8.20 ppm, 8.25 ppm, 10.19 ppm, 11.73 ppm, and 11.84 ppm.

Some embodiments comprise a method of making crystalline Form C anhydrate as an anhydrate of Compound 1. The method of making crystalline Form C anhydrate comprises admixing acetonitrile and crystalline Form A hydrate to form a slurry. The method also comprises stirring the solution at about 40° C. for about 1 week to obtain a slurry. The method comprises where the slurry comprises crystalline Form C anhydrate. Some embodiments of the method comprise crystalline Form C anhydrate is separated from the slurry and dried or allowed to dry to form crystalline Form C anhydrate.

Some embodiments comprise a crystalline Form C anhydrate that is substantially free of crystalline Form A hydrate, and compositions comprising crystalline Form C anhydrate that are substantially free of crystalline Form A hydrate. In some embodiments, the composition has less than about 4% (w/w) of crystalline Form A hydrate. Some embodiments comprise a composition comprising crystalline Form C anhydrate that is substantially free of crystalline Form A hydrate, wherein the composition has less than about 2% (w/w) of crystalline Form A hydrate. Some embodiments comprise making a composition comprising an anhydrate of crystalline Form C anhydrate that is substantially free of the hydrate crystalline Form A hydrate.

Some embodiments comprise a method of making Compound 1 crystalline Form C anhydrate. The method of making crystalline Form C anhydrate also comprises admixing absolute ethanol and Compound 1 crystalline Form A hydrate to form a slurry. The method comprises stirring the slurry at about 40° C. for about two days. The method comprises where the slurry comprises crystalline Form C anhydrate. Some embodiments of the method comprise crystalline Form C anhydrate is separated from the slurry and dried or allowed to dry to form crystalline Form C anhydrate.

In some embodiments, provided herein are methods for making a Compound 1 crystalline Form C anhydrate, wherein the Compound 1 crystalline Form C anhydrate is substantially free of Compound 1 crystalline Form A hydrate.

Some embodiments comprise the composition of crystalline Form C anhydrate where the composition is at least about 1-75% % by weight crystalline Form C anhydrate inclusive, comprising embodiments where the composition is about 1% by weight crystalline Form C anhydrate, or about 5% by weight crystalline Form C anhydrate, for example. Said composition may comprise at least one pharmaceutically acceptable carrier or diluent.

Some embodiments comprise a method of making Compound 1 crystalline Form C anhydrate. The method of making crystalline Form C anhydrate comprises admixing absolute ethanol and crystalline Form A hydrate to form a slurry. The method comprises stirring the slurry at about 50° C. for about one hour. The method also comprises where the slurry comprises crystalline Form C anhydrate. Some embodiments of the method comprise crystalline Form C anhydrate is separated from the slurry and dried or allowed to dry to form crystalline Form C anhydrate.

Some embodiments comprise a Compound 1 crystalline Form E DMSO solvate.

Some embodiments comprise crystalline Form E DMSO solvate having an X-ray powder diffraction pattern substantially as shown in FIG. 20.

In some embodiments, crystalline Form E DMSO solvate is characterized by an X-ray powder diagram having characteristic reflections at the following d values: 14.7791 Å, 13.6398 Å, 10.2578 Å, 9.7391 Å, and 7.4243 Å. In some embodiments, crystalline Form E DMSO solvate is further characterized by in an X-ray powder diffraction pattern that may comprise a peak at each of diffraction angles (2θ): 6.0±0.2°, 6.5±0.2°, 8.6±0.2°, 9.1±0.2°, 11.9±0.2°. In some embodiments, crystalline Form E DMSO solvate is further characterized by in an X-ray powder diffraction pattern that may comprise a peak at each of diffraction angles (2θ): 6.0±0.2°, 6.5±0.2°, and 8.6±0.2°.

In some embodiments, crystalline Form E DMSO solvate is characterized as having a solution 1H NMR spectrum in DMSO-d6 that may comprise one or more peaks at about 2.53 ppm, 3.58 ppm, 4.05 ppm, 4.38 ppm, 4.65 ppm, 4.91 ppm, 7.07 ppm, 7.22 ppm, 7.29 ppm, 7.43 ppm, 7.48 ppm, 7.60 ppm, 7.68 ppm, 7.74 ppm, 7.90 ppm, 8.09 ppm, 8.20 ppm, 8.25 ppm, 10.19 ppm, 11.73 ppm, and 11.84 ppm.

Some embodiments comprise a method of making crystalline Form E DMSO solvate of Compound 1. The method of making crystalline Form E DMSO solvate comprises admixing DMSO and crystalline Form A hydrate to form a solution. The method comprises adding water dropwise to form a slurry and stirring the slurry for about one day. The method comprises where the slurry comprises crystalline Form E DMSO solvate. Some embodiments of the method comprise the crystalline Form E DMSO solvate is separated from the slurry and dried or allowed to dry to form crystalline Form E DMSO solvate.

Some embodiments may comprise one or more of the following features: the composition where the composition is about 1-75% % by weight crystalline Form E DMSO solvate inclusive, comprising embodiments where the composition is about 1% by weight crystalline Form E DMSO solvate, or about 5% by weight crystalline Form E DMSO solvate, for example. Said composition may comprise at least about one pharmaceutically acceptable carrier or diluent.

Some embodiments comprise a crystalline Form F DMA solvate of Compound 1.

Some embodiments comprise crystalline Form F DMA solvate having an X-ray powder diffraction pattern substantially as shown in FIG. 22.

In some embodiments, crystalline Form F DMA solvate is characterized by an X-ray powder diagram that has characteristic reflections at the following d values: 11.3925 Å, 10.9421 Å, 9.7391 Å, 9.3487 Å, and 8.9342 Å.

In some embodiments, crystalline Form F DMA solvate is further characterized by in an X-ray powder diffraction pattern that may comprise a peak at each of diffraction angles (2θ): 7.8±0.2°, 8.1±0.2°, 9.1±0.2°, 9.5±0.2°, and 9.9±0.2°.

In some embodiments, crystalline Form F DMA solvate further characterized by in an X-ray powder diffraction pattern that may comprise a peak at each of diffraction angles (2θ): 8.1±0.2°, 9.1±0.2°, and 9.9±0.2°.

Some embodiments comprise a method of making crystalline Form F as a DMA solvate of Compound 1. The method of making crystalline Form F DMA solvate comprises admixing a about 70:about 30 DMA:H₂O and a mixture of crystalline Form A hydrate and crystalline Form C anhydrate to form a slurry, stirring the slurry for about one week, wherein the slurry comprises crystalline Form F DMA solvate, where the slurry is dried or allowed to dry to form crystalline Form F DMA solvate. In some embodiments, the DMA is dry DMA.

In some embodiments, crystalline Form F DMA solvate is characterized as having a solution ¹H NMR spectrum that may comprise one or more peaks at about 1.95 ppm, 2.78 ppm, 2.94 ppm, 3.58 ppm, 4.05 ppm, 4 . . . 38 ppm, 4.65 ppm, 4.91 ppm, 7.07 ppm, 7.22 ppm, 7.29 ppm, 7.43 ppm, 7.48 ppm, 7.60 ppm, 7.68 ppm, 7.74 ppm, 7.90 ppm, 8.09 ppm, 8.20 ppm, 8.25 ppm, 10.19 ppm, 11.73 ppm, and 11.84 ppm.

Other embodiments may comprise one or more of the following features: the composition where the composition is about 1-75% % by weight crystalline Form F DMA solvate inclusive, comprising embodiments where the composition is about 1% by weight crystalline Form F DMA solvate, and about 5% by weight crystalline Form F DMA solvate, for example. Said composition may comprise at least one pharmaceutically acceptable carrier or diluent.

In some embodiments, a method for treating cancer is described.

In some embodiments, the methods for treating cancer reduce the size of a mammal's tumor. In some embodiments, the methods inhibit metastasis of a mammal's tumor. In some embodiments, the methods inhibit tumor growth. In some embodiments, the methods inhibit metastasis of a mammal's tumor. In some embodiments, the methods relieve or eliminate one or more symptoms associated with cancer.

### Definitions

As used herein, the term “about” when used in connection with a numerical value will be understood by persons of skill in the art to mean up to plus or minus 10% of the numerical value.

The term “active agent” or “active ingredient” refers to a Compound 1 crystalline Form, a mixture comprising more than one Compound 1 crystalline Form or a mixture comprising a Compound 1 crystalline Form and an amorphous form of Compound 1.

As used herein, the terms “substantially pure” and “substantially free” with reference to a particular polymorphic form (or to a mixture of two or more polymorphic forms) of Compound 1 indicates the polymorphic form (or a mixture) comprises less than 10%, less than 5%, less than 3%, less than 1% by weight of impurities, comprising other polymorphic forms of Compound 1. Such purity may be determined, for example, by powder X-ray diffraction.

As used herein, the term “room temperature” refers to a temperature condition typically encountered in a laboratory setting. This comprises the approximate temperature range of about 18 to about 30° C.

As used herein, the term “detectable amount” refers to an amount or amount per unit volume that can be detected using conventional techniques, such as X-ray powder diffraction, differential scanning calorimetry, HPLC, Fourier Transform Infrared Spectroscopy (FT-IR), Raman spectroscopy, and the like.

The term “pharmaceutical composition” refers to a composition comprising a polymorphic form of Compound about 1 described herein, and another chemical component, such as physiologically/pharmaceutically acceptable carriers, diluents, vehicles and/or excipients.

As used herein, the term “effective amount” refers to the amount of an active agent that is effective in treating cancer in a mammal. In some embodiments, the mammal is a human.

The terms listed in Table 1 are defined herein as:

TABLE 1
List of terms

| Abbreviation | Definition |
|---|---|
| DSC | Differential scanning calorimetry |
| DVS | Dynamic vapor sorption |
| FT-Raman | Fourier transform Raman |
| IR | Infrared |
| ¹H NMR | Proton nuclear magnetic resonance |
| KF | Karl Fischer |
| TG | Thermogravimetry |
| XRPD | X-ray powder diffraction |
| DMF | Dimethylformamide |
| ACN | Acetonitrile |
| DMSO | Dimethyl sulfoxide |
| DMA | Dimethylacetamide |

Novel Compound 1 crystalline Forms are described herein. Compound 1, and methods of making it, are described in U.S. Pat. No. 9,586,974 which is herein incorporated by reference in its entirety.

It has been found, as described herein, that Compound 1 can exist in multiple crystalline forms. These forms may be used, optionally in a composition in which a Compound 1 crystalline form is dissolved, for the treatment of cancer. Each form may have advantages over the others in terms of properties such as bioavailability, stability, and manufacturability. Novel crystalline forms of Compound 1 have been discovered which are likely to be more suitable for bulk preparation and handling than other forms. Also described herein are processes for the preparation of polymorphic forms of Compound 1 that are substantially free from other polymorphic forms of Compound 1.

In some embodiments, the solid crystalline Forms may also comprise more than one polymorphic crystalline Form. One of skill in the art will also recognize that crystalline forms of a given compound can exist in substantially pure forms of a single polymorph, but can also exist in a crystalline form that comprises a mixture of two or more different polymorphs or amorphous forms. Where a solid crystalline Form comprises two or more polymorphs, the X-ray diffraction pattern will typically have peaks characteristic of each of the individual polymorphs. For example, a solid crystalline Form that comprises two polymorphs will typically have a powder X-ray diffraction pattern that is a convolution of the two X-ray diffraction patterns that correspond to the substantially pure polymorphic crystalline Forms. For example, a solid crystalline Form of Compound 1 can comprise a first, second, third, fourth or fifth polymorphic crystalline Form where the solid crystalline Form comprises at least 10% by weight of the first polymorph. In a further example, the solid crystalline Form can comprise at least 20% by weight of the first polymorph. Even further examples comprise at least 30%, at least 40%, or at least 50% by weight of the first polymorph. One of skill in the art will recognize that many such combinations of several individual polymorphs and crystalline forms in varying amounts are possible.

### II. Methods for Treating Cancer

The disclosure also provides methods for treating cancer in a mammal in need thereof, comprising the step of administering to the mammal an effective amount of a Compound 1 crystalline Form. The Compound 1 crystalline Form can be crystalline Form A hydrate, crystalline Form B DMF solvate, crystalline Form C anhydrate, crystalline Form E DMSO solvate, crystalline Form F DMA solvate, or combinations thereof.

In some embodiments, the cancer is a solid-tumor cancer. In some embodiments, the cancer is a blood cancer.

In some embodiments, the cancer is a solid-tumor cancer, and the solid-tumor cancer is fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon cancer, colorectal cancer, kidney cancer, pancreatic cancer, bone cancer, breast cancer, ovarian cancer, prostate cancer, esophogeal cancer, stomach cancer, oral cancer, nasal cancer, throat cancer, squamous cell carcinoma, basal cell carcinoma, adenocarcinoma, sweat gland carcinoma, sebaceous gland carcinoma, papillary carcinoma, a papillary adenocarcinoma, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, uterine cancer, testicular cancer, small cell lung carcinoma, bladder carcinoma, lung cancer, epithelial carcinoma, glioma, glioblastoma multiforme, astrocytoma, medulloblastoma, craniopharyngioma, ependymoma, pinealoma, hemangioblastoma, acoustic neuroma, oligodendroglioma, meningioma, skin cancer, melanoma, neuroblastoma, retinoblastoma, or hepatocellular carcinoma.

In some embodiments, the cancer is a blood cancer, and the blood cancer is leukemia (e.g., an acute leukemia or a chronic leukemia, in either case, e.g., lymphoblastic leukemia, myelogenous leukemia, lymphocytic leukemia, myelocytic leukemia, acute lymphoblastic B-cell leukemia, acute lymphoblastic T-cell leukemia, acute myeloblastic leukemia, acute promyelocytic leukemia, acute monoblastic leukemia, acute erythroleukemic leukemia, acute megakaryoblastic leukemia, acute myelomonocytic leukemia, acute nonlymphocyctic leukemia, acute undifferentiated leukemia, chronic myelocytic leukemia, chronic lymphocytic leukemia, or hairy cell leukemia); lymphoma (e.g., Hodgkin's disease, non-Hodgkin's lymphoma, Waldenstrom's macroglobulinemia, heavy chain disease, or polycythemia vera); or myeloma (e.g., solitary plasmacytoma, extramedullary plasmacytoma or multiple myeloma).

It will be appreciated that the actual dosages of Compound 1 crystalline Forms useful in the compositions of this disclosure can vary according to the particular polymorphic form being used, the particular composition formulated, the mode of administration and the particular site, host and disease being treated. Those skilled in the art using conventional dosage-determination tests in view of the experimental data for an agent can ascertain optimal dosages for a given set of conditions. That level is typically an amount sufficient to provide about 100 to about 3000 μg/kg of body weight to the recipient's plasma or serum.

For oral administration, an illustrative daily dose useful for treating cancer is from about 0.001 to about 100 mg/kg of body weight, in one embodiment from about 0.01 to about 50 mg/kg body weight, and courses of treatment can be repeated at appropriate intervals. In the practice of the disclosure, the most suitable route of administration as well as the magnitude of a therapeutic dose can depend on the nature and severity of the disease to be treated. The dose, and dose frequency, may also vary according to the age, body weight, and response of the individual patient. A suitable oral dosage form may cover a dose range from 0.5 mg to 100 mg of active ingredient total daily dose, administered in one single dose or equally divided doses. In one embodiment the amount of the Compound 1 crystalline Form in such formulations is from about 0.5 mg to about 20 mg, such as from about 1 mg to about 10 mg or from about 1 mg to about 5 mg.

### III. Pharmaceutical Compositions

The disclosure also provides methods for treating cancer in a mammal in need thereof, comprising the step of administering to the mammal an effective amount of a composition in which a Compound 1 crystalline Form of Compound 1 is dissolved, e.g., in a suitable solvent disclosed herein.

In some embodiments, the administering is parenteral; in some embodiments, the administering is intravenous.

The active agents (e.g., the crystalline forms or solid forms comprising two or more such forms, of Compound 1) described herein may be formulated into pharmaceutical compositions suitable for mammalian medical use. Any suitable route of administration may be employed for providing a patient with an effective dosage of any of the polymorphic forms of Compound 1. For example, peroral or parenteral compositions and the like may be employed. Dosage forms comprise capsules, tablets, dispersions, suspensions and the like, e.g. enteric-coated capsules and/or tablets, capsules and/or tablets comprising enteric-coated pellets of polymorphic forms of Compound 1. In all dosage forms, polymorphic forms of Compound 1 can be admixed with other suitable constituents. The compositions may be conveniently presented in unit dosage forms, and prepared by any methods known in the pharmaceutical arts. Pharmaceutical compositions of the disclosure typically comprise an effective amount of the active agent and one or more inert, pharmaceutically acceptable carriers, and optionally any other therapeutic ingredients, stabilizers, or the like. The carrier(s) are typically pharmaceutically acceptable in the sense of being compatible with the other ingredients of the composition and not unduly deleterious to the recipient thereof. The compositions may further comprise diluents, buffers, binders, disintegrants, thickeners, lubricants, preservatives (comprising antioxidants), flavoring agents, taste-masking agents, inorganic salts (e.g., sodium chloride), antimicrobial agents (e.g., benzalkonium chloride), sweeteners, antistatic agents, surfactants (e.g., polysorbates such as “TWEEN™ 20” and “TWEEN™ 80”, and pluronics such as F68 and F88, available from BASF), sorbitan esters, lipids (e.g. long chain, medium chain, short chain and mixtures thereof), phospholipids such as lecithin and other phosphatidylcholines, phosphatidylethanolamines, fatty acids and fatty esters, steroids (e.g., cholesterol)), and chelating agents (e.g., EDTA, zinc and other such suitable cations). Other pharmaceutical excipients and/or additives suitable for use in the compositions according to the present disclosure are listed in Remington: The Science & Practice of Pharmacy, 19th ed., Williams & Williams, (1995), and in the “Physician's Desk Reference”, 52nd ed., Medical Economics, Montvale, NJ (1998), and in Handbook of Pharmaceutical Excipients, 3rd. Ed., Ed. A. H. Kibbe, Pharmaceutical Press, 2000. The active agents of the present disclosure may be formulated in compositions comprising those suitable for oral, rectal, topical, nasal, ophthalmic, or parenteral (comprising intraperitoneal, intravenous, subcutaneous, or intramuscular injection) administration.

The amount of the active agent in the composition can vary depending upon a variety of factors, comprising dosage form, the condition to be treated, target patient population, and other considerations, and will be determined by one skilled in the art. In practice, this can vary widely depending, for example, upon the particular active agent, the severity of the condition to be treated, the patient population, the stability of the composition, and the like. Compositions will comprise anywhere from about 0.001% by weight to about 99% by weight active agent, in some embodiments from about 0.01% to about 5% by weight active agent, and in some embodiments from about 0.01% to 2% by weight active agent, and can also depend upon the relative amounts of excipients/additives comprised in the composition.

In some embodiments, a pharmaceutical composition can be administered in conventional dosage form prepared by combining an effective amount of an active agent as an active ingredient with one or more appropriate pharmaceutical carriers according to conventional procedures. These procedures may involve mixing, granulating and compressing or dissolving the ingredients as appropriate to the desired preparation.

The pharmaceutical carrier(s) employed may be either solid or liquid. Exemplifying solid carriers comprise, but are not limited to, lactose, sucrose, talc, gelatin, agar, pectin, acacia, magnesium stearate, stearic acid and the like. Exemplifying liquid carriers comprise syrup, peanut oil, olive oil, water and the like. Similarly, the carrier(s) may comprise time-delay or time-release materials known in the art, such as glyceryl monostearate or glyceryl distearate alone or with a wax, ethylcellulose, hydroxypropylmethylcellulose, methylmethacrylate and the like.

A variety of pharmaceutical forms can be employed. For example, if a solid carrier is used, the preparation can be tableted, placed in a hard gelatin capsule in powder or pellet form or in the form of a troche or lozenge. The amount of solid carrier may vary, but will be from about 25 mg to about 1 g. If a liquid carrier is used, the preparation can be in the form of syrup, emulsion, soft gelatin capsule, sterile injectable solution or suspension in an ampoule or vial or non-aqueous liquid suspension.

The active agent may be dissolved in a suitable co-solvent or combinations of co-solvents. Examples of suitable co-solvents comprise, but are not limited to, alcohol, propylene glycol, polyethylene glycol 300, polysorbate 80, gylcerin and the like in concentrations ranging from about 0 to about 60% of the total volume.

It will be appreciated that the actual dosages of Compound 1 crystalline Forms useful in the compositions of this disclosure can vary according to the particular polymorphic form being used, the particular composition formulated, the mode of administration and the particular site, host and disease being treated. Those skilled in the art using conventional dosage-determination tests in view of the experimental data for an agent can ascertain optimal dosages for a given set of conditions. That level is typically an amount sufficient to provide about 100 to about 3000 μg/kg of body weight to the recipient's plasma or serum.

For oral administration, an illustrative daily dose employed is from about 0.001 to about 100 mg/kg of body weight, in some embodiments from about 0.01 to about 50 mg/kg body weight, and courses of treatment can be repeated at appropriate intervals. Administration of prodrugs is typically dosed at weight levels that are chemically equivalent to the weight levels of the fully active form. In the practice of the disclosure, the most suitable route of administration as well as the magnitude of a therapeutic dose will depend on the nature and severity of the disease to be treated. The dose, and dose frequency, may also vary according to the age, body weight, and response of the individual patient. A suitable oral dosage form may cover a dose range from 0.5 mg to 100 mg of active ingredient total daily dose, administered in one single dose or equally divided doses. In some embodiments the amount of the Compound 1 crystalline Form in such compositions is from about 0.5 mg to about 20 mg, such as from about 1 mg to about 10 mg or from about 1 mg to about 5 mg.

The compositions of the present disclosure may be manufactured in manners known for making pharmaceutical compositions, e.g., using conventional techniques such as mixing, dissolving, granulating, dragee-making, levigating, emulsifying, encapsulating, entrapping or lyophilizing. Pharmaceutical compositions may be formulated in a conventional manner using one or more physiologically acceptable carriers, which may be selected from excipients and auxiliaries that facilitate processing of the active compounds into preparations that can be used pharmaceutically.

For oral administration, a polymorphic form of Compound 1 can be formulated readily by combining the active agent with pharmaceutically acceptable carriers known in the art. Such carriers enable the compounds of the disclosure to be formulated as tablets, pills, dragees, capsules, gels, syrups, slurries, suspensions and the like, for oral ingestion by a patient to be treated. Pharmaceutical preparations for oral use can be obtained using a solid excipient in admixture with the active agent, optionally grinding the resulting mixture, and processing the mixture of granules after adding suitable auxiliaries, if desired, to obtain tablets or dragee cores. Suitable excipients comprise: fillers such as sugars, comprising lactose, sucrose, mannitol, or sorbitol; and cellulose preparations, for example, maize starch, wheat starch, rice starch, potato starch, gelatin, gum, methyl cellulose, hydroxypropylmethyl-cellulose, sodium carboxymethylcellulose, or polyvinylpyrrolidone (PVP). If desired, disintegrating agents may be added, such as crosslinked polyvinyl pyrrolidone, agar, or alginic acid or a salt thereof such as sodium alginate.

Dragee cores are provided with suitable coatings. For this purpose, concentrated sugar solutions may be used, which may optionally comprise gum arabic, polyvinyl pyrrolidone, Carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active agents.

Pharmaceutical preparations that can be used orally comprise push-fit capsules made of gelatin, as well as soft, sealed capsules made of gelatin and a plasticizer, such as glycerol or sorbitol. The push-fit capsules can comprise the active ingredients in admixture with fillers such as lactose, binders such as starches, and/or lubricants such as talc or magnesium stearate, and, optionally, stabilizers. In soft capsules, the active agents may be dissolved or suspended in suitable liquids, such as fatty oils, liquid paraffin, or liquid polyethylene glycols. In addition, stabilizers may be added. All compositions for oral administration should be in dosages suitable for such administration. For buccal administration, the compositions may take the form of tablets or lozenges formulated in conventional manner.

For administration intranasally or by inhalation, the compounds can be conveniently delivered in the form of an aerosol spray presentation from pressurized packs or a nebuliser, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of gelatin for use in an inhaler or insufflator and the like may be formulated comprising a powder mix of the compound and a suitable powder base such as lactose or starch.

The active agents may be formulated for parenteral administration by injection, e.g., by bolus injection or continuous infusion. Compositions for injection may be presented in unit-dosage form, e.g., in ampoules or in multi-dose containers, with an added preservative. The compositions may take such forms as suspensions, solutions or emulsions in oily or aqueous vehicles, and may comprise formulatory agents such as suspending, stabilizing and/or dispersing agents.

Compositions for intravenous administration can comprise a carrier selected from the group consisting of water soluble organic solvents, non-ionic surfactants, water insoluble lipids, organic lipids/semisolids and phospholipids. Water soluble organic solvents may be selected from, for example, polyethylene glycol 300, polyethylene glycol 400, ethanol, propylene glycol, glycerin, N-methyl-2-pyrrolidone, dimethylacetamide and dimethylsulfoxide. Non-ionic surfactants may be selected from Cremophor EL, Cremophor RH 40, Cremophor RH 60, d-α-tocopherol polyethylene glycol 1000 succinate, polysorbate 80, Solutol HS 15, sorbitan monooleate, poloxamer 407, Labrifil M-1944CS, Labrafil M-2125CS, Labrasol, Gellucire 44/14, Softigen 767, and mono- and di-fatty acid esters of PEG 300, 400 or 1750. The water insoluble lipids are selected from castor oil, corn oil, cottonseed oil, olive oil, peanut oil, peppermint oil, safflower oil, sesame oil, soybean oil, hydrogenated vegetable oils, hydrogenated soybean oil, and medium chain triglycerides of coconut oil and palm seed oil. Organic liquids and semisolids may be selected from beeswax, d-α-tocopherol, oleic acid and medium chain mono- and diglycerides. The phospholipids are selected from lecithin, hydrogenated soy phosphatidylcholine, distearoylphosphatidylglycerol, L-α-dimyristoylphosphatidylcholine and L-α-dimyristoylphosphatidylglycerol and others as disclosed herein.

Carriers comprise dimethylacetamide, dimethylsulfoxide, dimethylformamide, cyclodextrins and their derivatives, albumin, tonicity modifiers comprising buffers, salts, and glycerol; oils and lipids comprising medium and long chain triglycerides and mixtures thereof; natural and synthetic emulsifiers comprising phospholipids and phosphatidylcholine and mixtures thereof.

In the case of compositions for intravenous delivery, frequency of dosage for an active form of Compound 1 can be between 1 to 2 times per week to once every 4 weeks.

Pharmaceutical compositions for other parenteral administration comprise, for example, suspensions of the active agents and may be prepared as appropriate oily injection suspensions. Suitable lipophilic solvents or vehicles comprise fatty oils such as sesame oil, or synthetic fatty acid esters, such as ethyl oleate or triglycerides, or liposomes. Aqueous injection suspensions may comprise substances that increase the viscosity of the suspension, such as sodium carboxymethyl cellulose, sorbitol, or dextran. Optionally, the suspension may also comprise suitable stabilizers or agents that increase the solubility of the active agents to allow for the preparation of highly concentrated solutions.

For administration to the eye, the active agent can be delivered in a pharmaceutically acceptable ophthalmic vehicle such that the compound is maintained in contact with the ocular surface for a sufficient time period to allow the compound to penetrate the corneal and internal regions of the eye, comprising, for example, the anterior chamber, posterior chamber, vitreous body, aqueous humor, vitreous humor, cornea, iris/ciliary, lens, choroid/retina and sclera. The pharmaceutically acceptable ophthalmic vehicle may be, for example, an ointment, vegetable oil, or an encapsulating material. An active agent of the present disclosure may also be injected directly into the vitreous and aqueous humor or subtenon.

Alternatively, the active ingredient may be in powder form for constitution with a suitable vehicle, e.g., sterile pyrogen-free water, before use. The compounds may also be formulated in rectal or vaginal compositions such as suppositories or retention enemas, e.g., comprising conventional suppository bases such as cocoa butter or other glycerides.

In addition to the compositions described above, the polymorphic forms may also be formulated as a depot preparation. Such long-acting compositions may be administered by implantation (for example, subcutaneously or intramuscularly) or by intramuscular injection. Thus, for example, the polymorphic forms may be formulated with suitable polymeric or hydrophobic materials (for example, as an emulsion in an acceptable oil) or ion-exchange resins, or as sparingly soluble derivatives, for example, as a sparingly soluble salt.

Additionally, polymorphic forms of Compound 1 may be delivered using a sustained-release system, such as semipermeable matrices of solid hydrophobic polymers comprising the therapeutic agent. Various sustained-release materials have been established and are known by those skilled in the art. Sustained-release capsules may, depending on their chemical nature, release the compound for a few weeks up to over 100 days.

The pharmaceutical compositions also may comprise suitable solid- or gel-phase carriers or excipients. Examples of such carriers or excipients comprise calcium carbonate, calcium phosphate, sugars, starches, cellulose derivatives, gelatin, and polymers such as polyethylene glycols.

### IV. Methods of Making the Polymorphs

### EXAMPLES

The examples which follow will further illustrate the preparation and characterization of the distinct polymorphic forms of Compound 1, but are not intended to limit the scope of the disclosure as described herein or as claimed herein. Unless otherwise indicated, all temperatures are set forth in degrees Celsius and all parts and percentages are by weight.

### Example 1: Compound 1 Crystalline Form A Hydrate Preparation

251 mg of Compound 1 were placed in an 8 mL glass vial followed by 2.0 mL of about 95:about 5 methanol:water (v/v). The resulting slurry was stirred magnetically at 40° C. for about two days. The slurry was vacuum filtered and the solids allowed to air dry for 15 minutes at room temperature.

### Example 2: Compound 1 Crystalline Form B DMF Solvate Preparation

19.8 mg of crystalline Form A hydrate were transferred to a vial and 1 mL of DMF was added. The resultant mixture was stirred magnetically at room temperature. The mixture was initially a clear solution but solids were formed after several hours. The resultant slurry was centrifuged and solids were collected and air dried overnight.

### Example 3: Compound 1 Crystalline Form C Anhydrate Preparation

20.2 mg of crystalline Form A hydrate were transferred to a vial. Aliquots adding up to 16 mL of acetonitrile were added. The resultant mixture was stirred magnetically at 40° C. for about 1 week. Once sufficient solids were formed, the resultant slurry was centrifuged and the solids were collected and dried in a vacuum desiccator for a few hours.

### Example 4: Compound 1 Crystalline Form E DMSO Solvate Preparation

20.3 mg of crystalline Form A hydrate was transferred to a vial. A volume of 1 mL of DMSO was added and the resultant mixture was stirred magnetically at room temperature. Water was added dropwise to the resultant solution, which became turbid after a few drops. The turbid mixture was kept on the stir plate at room temperature for about one day. Once sufficient solids were formed, the resultant slurry was centrifuged and the solids were collected and dried in a vacuum desiccator for a few hours.

### Example 5: Compound 1 Crystalline Form F DMA Solvate Preparation

12.4 mg of each crystalline Form A hydrate and crystalline Form C anhydrate were weighed into a vial and 0.5 mL of about 70:about 30 dry DMA:H2O was added. The mixture was stirred magnetically at room temperature for about 1 week. The resultant slurry was centrifuged and solids were collected and dried in a vacuum desiccator for a few hours.

### Example 6: Characterization of an Additional XRPD Peak in Crystalline Form A Hydrate

In one sample of Compound 1 crystalline Form A hydrate, a small broad peak at about 7.1° 2θ was observed in the XRPD pattern. The nature of this peak is unknown. The Compound 1 crystalline Form A hydrate was slurried in both methanol (FIG. 24A) and about 95:about 5 methanol:water (v/v) (FIG. 24B) at room temperature for about two days to determine whether the peak would disappear (Table 22). In both solvent systems at small scale (about 30 mg), the XRPD pattern of the material remained that of the crystalline Form A hydrate and the peak at 7.1° 20 remained. In the case of a larger scale experiment (about 250 mg) in about 95:about 5 methanol:water (v/v), the peak did become smaller (FIG. 24C). NMR data does not show evidence of an impurity.

TABLE 22
Slurry Experiments Using Compound 1 Crystalline Form A hydrate

| Sample | Experiment | XRPD Result |
|---|---|---|
| 1 | Slurry, 40° C., MeOH, two days, 30 mg | A, FIG. 24A |
| 2 | Slurry, 40° C., 95:5 MeOH:water, two | A, FIG. 24B |
| | days, 30 mg | |
| 3 | Slurry, 40° C., 95:5 MeOH:water, two | A, FIG. 24C |
| | days, 250 mg | |

### Example 7: Solid State Stability of Compound 1 Crystalline Form A Hydrate, Compound 1 Crystalline Form C Anhydrate and Amorphous Compound 1

The relative stability of the crystalline Form A hydrate and Compound 1 crystalline Form C anhydrate was evaluated in ethanol and ethanol/water between ambient temperature and 50° C. The results showed that the crystalline Form A hydrate converted to the crystalline Form C anhydrate except when the water activity of the solvent was greater than 0.31. Crystalline Form A hydrate converted to crystalline Form C anhydrate in ethanol at about 50° C. when analyzed after slurrying for four days.

Attempts to prepare crystalline Form C anhydrate and amorphous material are summarized in Table 23. Crystalline Form C anhydrate was prepared by slurrying crystalline Form A hydrate in absolute ethanol at 40° C. for about two days (Sample 1, FIG. 25A). Attempts to prepare amorphous material by cold precipitation and lyophilization were carried out but were unsuccessful. Two of the precipitation experiments did not produce solids, and the other two produced material having XRPD patterns consistent with crystalline Forms A and F, respectively (Sample 2, FIG. 25B; Sample 4, FIG. 25D). The lyophilization experiment produced material having an XRPD pattern consistent with crystalline Form E DMSO solvate (FIG. 25C).

TABLE 23
Preparation of Compound 1 Crystalline
Forms A, C, and Amorphous

| Sample | Experiment^{a} | XRPD result^{b} |
|---|---|---|
| 1 | SL, 40° C., EtOH, two days, | Crystalline Form C |
| | 250 mg | anhydrate, FIG. |
| | | 25A |
| 2 | P, DMAc/water | Crystalline Forms |
| | | F + A, FIG. 25B |
| 3 | P, DMF/acetone | — |
| 4 | P, DMF/Et₂O | Crystalline Form A |
| | | hydrate, FIG. 25D |
| 5 | P, DMF/DCM | — |
| 6 | Lyophilization, DMSO | Crystalline Form E |
| | | DMSO solvate |
| | | (LC), FIG. 25C |

^{a}DCM = dichloromethane; DMF = N,N-dimethylformamide; DMAc = dimethylacetamide; DMSO = dimethylsulfoxide; P = precipitation; SL = slurry
^{b}LC = low crystallinity

Crystalline Form A hydrate was prepared according to Example 1.

The DSC curve for the crystalline Form A hydrate exhibited a broad endotherm with signal maximum about 96° C. (FIG. 26). The TG curve exhibits a corresponding weight loss of 2.8% up to 100° C. which corresponds to about 0.94 moles of water per mole of Compound 1 crystalline Form A hydrate. This is consistent with the thermal data for crystalline Form A hydrate from polymorph screening which exhibits a broad DSC endotherm below 100° C. and TG weight loss of about 2.6%.

To prepare the crystalline Form C anhydrate, 249 mg of Compound 1 crystalline Form A hydrate was placed in an 8 mL glass vial followed by 2.0 mL of absolute ethanol. The resulting slurry was stirred magnetically at 40° C. for about two days. The slurry was vacuum filtered and the solids allowed to air dry for 15 minutes at room temperature.

The DSC curve for the crystalline Form C anhydrate exhibits no events below the decomposition point of about 275° C. (FIG. 27). The TG curve exhibits a small weight loss of about 0.8% up to 250° C. suggesting it is anhydrous. This is consistent with the thermal data for crystalline Form C anhydrate from the polymorph screening report. Compound 1 crystalline Form C anhydrate is unsolvated.

TABLE 24
Characterization of Compound 1 Crystalline Forms A and C

| Sample | Experiment^{a} | Results | FIGS. |
|---|---|---|---|
| 1 | DSC | Broad endo 96.3° C. | FIG. 26 |
| (Crystalline | TG | 2.8% start to 100° C. | FIG. 26 |
| Form A | NMR, | Consistent with structure | FIGS. 28A- |
| hydrate) | (DMSO-d6) | | 28C |
| 2 | DSC | No events below | FIG. 27 |
| (Crystalline | | decomposition point | |
| Form C | | (~275° C.) | |
| anhydrate) | TG | 0.8% start to 250° C. | FIG. 27 |
| | NMR, | Consistent with structure | FIGS. 29A- |
| | (DMSO-d6) | Residual ethanol (about 0.1 | 29C |
| | | moles) | |

### Example 8: Stability Studies of Crystalline Form A Hydrate and Crystalline Form C Anhydrate

A sample of each of crystalline Forms A and C was placed in an uncapped vial and exposed to 25° C./60% RH and 40° C./75% relative humidity (“RH”) for 4 weeks (Table 24). The resultant samples were analyzed by XRPD at T=1, 2, and 4 weeks to evaluate their physical stability. No crystalline Form conversion was observed after 4 weeks.

TABLE 25
Stability Study of Crystalline Form A hydrate and C

| [symbol] rting | | | Time | |
|---|---|---|---|---|
| [symbol] terial | Sample ID | Conditions | (weeks) | XRPD result |
| 1292-1-4 | 1292-5-1 | 25° C./60% RH | 1 | A |
| (Crystalline | | | | |
| Form A | | | | |
| hydrate) | | | | |
| | 1292-5-2 | | 2 | A |
| | 1292-5-3 | | 4 | A |
| | 1292-5-4 | 40° C./75% RH | 1 | A |
| | 1292-5-5 | | 2 | A |
| | 1292-5-6 | | 4 | A |
| 1292-1-3 | 1292-5-7 | 25° C./60% RH | 1 | C |
| (Crystalline | | | | |
| Form C | | | | |
| anhydrate) | | | | |
| | 1292-5-8 | | 2 | C |
| | 1292-5-9 | | 4 | C |
| | 1292-5-10 | 40° C./75% RH | 1 | C |
| | 1292-5-11 | | 2 | C |
| | 1292-5-12 | | 4 | C |

[symbol]

indicates data missing or illegible when filed

### Example 9: Conversion Rate of Crystalline Form A Hydrate to Crystalline Form C Anhydrate

The conversion rate of the crystalline Form A hydrate to the crystalline Form C anhydrate in ethanol at about 50° C. was investigated (Table 25). Slurries were prepared using Compound 1 crystalline Form A hydrate as starting material. Each slurry comprised about 20 mg of Compound 1 crystalline Form A hydrate and 1 mL of absolute ethanol. Each slurry was stirred magnetically at about 50° C. and subsequently vacuum filtered to collect the solid at given times. The resulting solids were analyzed by XRPD immediately. At T=1 hour the sample had converted to crystalline Form C anhydrate completely (FIG. 4).

TABLE 26
Conversion Rate of Crystalline Form A hydrate to
Crystalline Form C anhydrate in Ethanol at 50° C.

| Starting | | Time | |
|---|---|---|---|
| Material | Sample ID | (hours) | XRPD result |
| TCL18445 | 1292-2-1 | 1 | C |
| (Crystalline | | | |
| Form A | | | |
| hydrate) | | | |
| | 1292-2-2 | 2 | C |
| | 1292-2-3 | 5 | C |

### Example 10: Instrumentation

### A. X-Ray Powder Diffraction (XRPD)

A Rigaku SmartLab X-Ray Diffractometer was configured in Bragg-Brentano reflection geometry equipped with a beam stop and knife edge to reduce incident beam and air scatter. No filter was used for crystalline Form C anhydrate crystalline Form E DMSO solvate. Spinning rate of 17 rpm was used for crystalline Form C anhydrate analysis. Other parameters are the same as described in Table 27.

TABLE 27
XRPD Parameters.

| Parameter | Value |
|---|---|
| Geometry | Bragg-Brentano |
| Tube Anode | Cu |
| Tube Type | Long Fine Focus |
| Tube Voltage (kV) | 40 |
| Tube Current (mA) | 44 |
| Detector | D/teX Ultra 250 or |
| | HyPix-3000 |
| Monochromator | Ni foil Cu Kβ Filter |
| Incident Slit (°) | ⅓ |
| Receiving Slit 1 (mm) | 18 |
| Receiving Slit 2 (mm) | 20.1 (open for |
| | Crystalline Form A |
| | hydrate + Form B |
| | DMF solvate) |
| Start Angle 2θ (°) | 2 |
| End Angle 2θ (°) | 40 |
| Step Size (°) | 0.02 |
| Scan Speed (°/min) | 6 |
| Spinning (rpm) | 11 |
| Sample Holder | Low-background |
| | Si |

### B. Crystalline Forms of Compound 1

Several crystalline forms of Compound 1 are described herein. Each crystalline form can be characterized by one or more of the following: powder X-ray diffraction pattern (e.g., X-ray diffraction peaks at various diffraction angles (2θ)); ¹H NMR spectroscopy; melting point onset (and onset of dehydration for hydrated forms) as illustrated by endotherms of a Differential Scanning calorimetry (DSC) thermogram; thermal stability as illustrated by thermogravimetric analysis (TG), hygroscopic properties as illustrated by Dynamic Vapor Sorption (DVS) measurements; IR spectral diagram pattern; Raman spectral diagram pattern; and physical and chemical storage stability according to methods known in the art or described herein.

One of skill in the art will appreciate that the peak positions (2θ) of XRPD will show some variability, typically as much as 0.1 to 0.2 degrees (2θ), depending, for example, on the solvents being used and/or on the apparatus being used to measure the diffraction. Accordingly, where peak positions (2θ) are reported, one of skill in the art will recognize that such numbers are intended to encompass such variability. Furthermore, where the polymorphs of the present disclosure are described as having a powder X-ray diffraction pattern essentially the same as that shown in a given FIG., the term “essentially the same” is also intended to encompass such variability in diffraction peak positions.

Further, one skilled in the art will appreciate that relative peak intensities will show inter-apparatus variability as well as variability due to the degree of crystallinity, orientation, prepared sample surface, the degree of purity of the sample being analyzed, and other factors known to those skilled in the art, and should be taken as qualitative measures only. The skilled person will also appreciate that measurements using a different wavelength will result in different shifts according to the Bragg equation—nλ=2d sin θ. Such further XRPD patterns generated by use of alternative wavelengths are considered to be alternative representations of the XRPD patterns of the crystalline materials of embodiments described herein and as such are within the scope of the present embodiments.

The different polymorphs of Compound about 1 described herein were characterized using a Rigaku SmartLab X-Ray Diffractometer that was configured in Bragg-Brentano reflection geometry, equipped with a beam stop and knife edge to reduce incident beam and air scatter. No filter was used for crystalline Form C anhydrate and crystalline Form E DMSO solvate. Spinning rate of 17 rpm was used for crystalline Form C anhydrate analysis. Other parameters are the same as described in Table 2.

TABLE 2
Parameters used for XRPD

| Parameter | Value |
|---|---|
| Geometry | Bragg-Brentano |
| Tube Anode | Cu |
| Tube Type | Long Fine Focus |
| Tube Voltage (kV) | 40 |
| Tube Current (mA) | 44 |
| Detector | D/teX Ultra 250 or |
| | HyPix-3000 |
| Monochromator | Ni foil Cu Kβ Filter |
| Incident Slit (°) | ⅓ |
| Receiving Slit 1 (mm) | 18 |
| Receiving Slit 2 (mm) | 20.1 (open for |
| | crystalline Forms |
| | A and B) |
| Start Angle 2θ (°) | 2 |
| End Angle 2θ (°) | 40 |
| Step Size (°) | 0.02 |
| Scan Speed (°/min) | 6 |
| Spinning (rpm) | 11 |
| Sample Holder | Low-background |
| | Si |

The different polymorphs of Compound 1 described herein can also be characterized using solid state NMR spectroscopy according to methods known in the art or described herein. For example, the ¹H NMR spectra were acquired on a Bruker Avance II 400 spectrometer. Samples were prepared by dissolving material in DMSO-d6. The solutions were placed into individual 5-mm NMR tubes for subsequent spectral acquisition. The temperature controlled (298K) ¹H NMR spectra acquired on the Avance II 400 utilized a 5-mm cryoprobe operating at an observing frequency of 400.18 MHz. Each spectrum was processed using TopSpin version 4.1.4 and referenced to the chemical shift of the residual DMSO-d6 (2.5 ppm) peak.

Different crystalline forms of Compound 1 were also distinguished using differential scanning calorimetry (DSC). DSC measures the difference in heat energy uptake between a sample and an appropriate reference with increase in temperature. For example, for the measurement of a solid powder sample, the reference can be an empty sample pan of the type used in preparation of the sample. DSC thermograms can be characterized by endotherms (indicating energy uptake) and also by exotherms (indicating energy release), typically as the sample is heated. Depending on several factors, the endotherms exhibited may vary by about 0.01-5° C. for crystal polymorphs melting above or below the endotherms, such as those depicted in the appended figures (FIGS.). Factors responsible for such variance comprise, for example, the rate of heating (e.g., the scan rate) at which the DSC analysis is conducted, the way the DSC onset temperature is defined and determined, the calibration standard used, instrument calibration, the relative humidity and the chemical purity of the sample. For any given sample, the observed endotherms may also differ from instrument to instrument; however, it will be within the ranges described herein provided the instruments are calibrated similarly.

The DSC analysis was carried out using a TA Instruments Q2500 Discovery Series instrument. The instrument temperature calibrations were performed using indium. The DSC cell was kept under a nitrogen purge of ~50 mL per minute during the analysis. The sample was placed in a standard, crimped aluminum pan and heated from approximately 25° C. to 250° C. at a rate of 10° C. per minute. Crystalline Form A hydrate was heated to 350° C.

Different polymorphic forms of a compound may have different hygroscopic properties. For example, crystalline Forms of Compound 1 were characterized based on their hygroscopic properties using dynamic vapor sorption measurements using a TA Instruments Q5000 Dynamic Vapor Sorption analyzer. A sample weight of less than 10 mg was loaded into a metal-coated quartz pan for analysis. The sample was analyzed at 25° C. after equilibrated to 5% relative humidity (RH) in 10% RH steps from 5 to 95% RH (adsorption cycle) and from 95 to 5% RH (desorption cycle). The movement from one step to the next occurred either after satisfying the equilibrium criterion of 0.01% weight change in 5 minutes or, if the equilibrium criterion was not met, after 90 minutes. The percent weight change values are calculated using Microsoft Excel®.

Five polymorphic crystalline Forms of Compound 1 have been identified and characterized as indicated in the FIGS. The crystalline Forms are designated polymorph crystalline Forms A, B, C, E and F. The polymorphs, pharmaceutical compositions comprising one or more polymorphs, and methods of using the polymorphs and pharmaceutical compositions thereof are described in more detail in the following sections and examples.

### A. Compound 1, Polymorph Form A Hydrate

Compound 1 crystalline Form A hydrate is a slightly hygroscopic, hydrated, crystalline material and is a variable hydrate, between a hemihydrate and a monohydrate.

KF analysis of Compound 1 crystalline Form A hydrate showed a water content of 2.4%.

Compound 1 crystalline Form A hydrate was successfully indexed, indicating it is a pure crystalline phase.

The ¹H NMR spectrum in DMSO-d6 shown in FIG. 8 is consistent with the chemical structure of Compound 1 crystalline Form A hydrate, with 0.03 mol DMF and 0.03 mol of hexane. This suggests that about 2.1% of the 2.6% weight loss observed in the TGA is due to water, which is 0.7 moles of water per molecule of Compound 1 crystalline Form A hydrate. ¹H NMR peak list in DMSO-d6 for Compound 1 crystalline Form A hydrate is shown in Table 3.

TABLE 3
¹H NMR peaks for Compound 1 crystalline Form A hydrate
Chemical Shift (ppm)

| | |
|---|---|
| 3.58 | (s) |
| 4.05 | (m) |
| 4.38 | (m) |
| 4.65 | (dd) |
| 4.91 | (t) |
| 7.07 | (t) |
| 7.22 | (t) |
| 7.29 | (s) |
| 7.43 | (d) |
| 7.48 | (t) |
| 7.60 | (dd) |
| 7.68 | (d) |
| 7.74 | (t) |
| 7.90 | (dd) |
| 8.09 | (s) |
| 8.20 | (dd) |
| 8.25 | (s) |
| 10.19 | (s) |
| 11.73 | (d) |
| 11.84 | (d) |

Compound 1 crystalline Form A hydrate was characterized by the XRPD pattern shown in FIG. 2. The XRPD pattern of Compound 1 crystalline Form A hydrate, is expressed in terms of the degree (20) and relative intensities, measured on a Rigaku SmartLab X-Ray Diffractometer. Table 4 shows the XRPD analysis of Compound 1 crystalline Form A hydrate, with select peaks unique to Compound 1 crystalline Form A hydrate shown in bold.

TABLE 4
XRPD analysis of Compound 1 crystalline Form A hydrate

| | Relative | d-spacing |
|---|---|---|
| °2θ ± 0.2 | Intensity | value (Å) |
| 6.1 | 5.08 | 14.4886 |
| 8.7 | 53.21 | 10.1636 |
| 9.7 | 33.56 | 9.1368 |
| 12.1 | 3.00 | 7.2903 |
| 13.7 | 18.71 | 6.4541 |
| 13.9 | 18.19 | 6.3709 |
| 14.6 | 5.30 | 6.0836 |
| 15.4 | 5.77 | 5.7388 |
| 15.8 | 7.63 | 5.6018 |
| 16.3 | 21.95 | 5.4312 |
| 17.6 | 5.04 | 5.0504 |
| 17.9 | 15.54 | 4.9443 |
| 18.3 | 6.21 | 4.8584 |
| 18.4 | 5.49 | 4.8269 |
| 18.9 | 6.57 | 4.6953 |
| 19.1 | 11.79 | 4.6514 |
| 19.4 | 48.12 | 4.5800 |
| 20.5 | 15.70 | 4.3407 |
| 21.5 | 23.43 | 4.1368 |
| 22.0 | 10.37 | 4.0329 |
| 22.3 | 10.36 | 3.9865 |
| 22.6 | 37.15 | 3.9342 |
| 23.0 ± 0.2 | 6.90 | 3.8601 |
| 23.4 ± 0.2 | 100.00 | 3.8015 |
| 25.0 ± 0.2 | 10.64 | 3.5561 |
| 25.4 ± 0.2 | 44.13 | 3.5065 |
| 25.9 ± 0.2 | 8.67 | 3.4400 |
| 26.2 ± 0.2 | 5.67 | 3.4013 |
| 26.6 ± 0.2 | 5.04 | 3.3560 |
| 27.0 ± 0.2 | 11.35 | 3.3071 |
| 27.6 | 9.77 | 3.2341 |
| 27.8 | 25.36 | 3.2045 |
| 28.4 | 6.06 | 3.1426 |
| 28.6 | 23.70 | 3.1168 |
| 29.2 | 15.30 | 3.0562 |
| 29.4 | 9.42 | 3.0339 |
| 29.7 | 7.39 | 3.0059 |
| 30.7 | 7.97 | 2.9122 |
| 30.8 | 9.43 | 2.8993 |
| 31.1 | 4.69 | 2.8720 |
| 31.3 | 4.37 | 2.8613 |
| 31.8 | 3.47 | 2.8105 |
| 32.2 | 3.34 | 2.7799 |
| 33.2 | 3.89 | 2.7016 |
| 33.5 | 3.68 | 2.6765 |
| 33.8 | 3.80 | 2.6519 |
| 34.2 | 4.29 | 2.6232 |
| 34.4 | 3.80 | 2.6084 |
| 34.6 | 3.78 | 2.5924 |
| 34.9 | 3.99 | 2.5736 |
| 34.9 | 3.25 | 2.5679 |
| 35.4 | 3.13 | 2.5328 |
| 36.5 | 4.30 | 2.4630 |
| 36.6 | 3.51 | 2.4577 |
| 37.0 | 2.61 | 2.4308 |
| 37.8 | 5.61 | 2.3811 |
| 37.9 | 4.32 | 2.3739 |
| 38.7 | 2.93 | 2.3266 |
| 39.3 | 4.01 | 2.2902 |
| 39.9 | 4.40 | 2.2572 |

For solid compositions comprising Compound 1 crystalline Form A hydrate, the solid compositions can be analyzed by XRPD to identify Compound 1 crystalline Form A hydrate in the product. This can be done by de-convoluting the XRPD data obtained from the formulated product. This can be achieved by subtracting the known excipient signals from the XRPD data of the formulated product. In addition, various chemometric techniques can be used to identify Compound 1 crystalline Form A hydrate in a solid composition, for example principal component analysis. Such analysis may be required to identify XRPD peaks belonging to Compound 1 Form A hydrate as they may overlap with XRPD peaks of some of the excipients used in the composition.

The DSC thermogram of Compound 1 crystalline Form A hydrate (FIG. 3) exhibits a broad endotherm about 78° C., which corresponds to the weight loss in the TG data. The material melts between 279-281° C. accompanied by decomposition events.

The TG thermogram of Compound 1 crystalline Form A hydrate (FIG. 4) exhibits a weight loss of 2.6% up to 70° C.

Additionally, Compound 1 crystalline Form A hydrate is slightly hygroscopic (FIG. 5), exhibiting a weight gain of less than 0.5% up to 95% relative humidity (RH). The weight gain is relative to the dehydrated crystalline Form A hydrate when exposed to the low RH at the start of DVS run. Equilibrium was not reached in the DVS data, suggesting that a weight gain of greater than 0.5% could be possible with longer equilibration time.

The infrared (FIG. 6) and Raman (FIG. 7) spectra are consistent with the chemical structure of Compound 1. Tables 5 and 6 show IR peaks and Raman shifts, respectively. In addition, multiple weak, broad peaks between 3420-3650 cm-1 indicate the presence of water, supporting the fact that crystalline Form A is a hydrate. Select peaks/shifts unique to Compound 1 crystalline Form A are shown in bold.

TABLE 5
Infrared peak list of Compound 1 Crystalline Form A hydrate
IR peak (cm⁻¹) ± 2

419
434
441
463
485
515
529
549
558
572
593
604
612
631
681
705
728
748
763
797
820
832
846
864
884
910
929
936
964
982
1012
1033
1048
1086
1119
1134
1142
1183
1226
1251
1272
1290
1309
1342
1405
1426
1465
1473
1512
1547
1579
1608
1629
1648
3250
3364
3495
3642

TABLE 6
Complete Raman shift list for Compound 1 Crystalline Form A hydrate.
Raman shift (cm⁻¹) ± 2

124
146
182
228
261
297
351
402
417
432
462
484
525
555
601
629
672
680
704
729
748
761
789
808
846
863
881
893
907
934
961
981
1009
1053
1083
1117
1139
1174
1191
1205
1233
1250
1272
1305
1340
1346
1376
1407
1443
1473
1512
1540
1578
1592
1620
1628
1653
2894
2909
2961
3003
3063
3144

For compositions comprising Compound 1 crystalline Form A hydrate, the compositions can be analyzed by IR and/or Raman spectroscopy to identify Compound 1 crystalline Form A hydrate in the product. This can be done by de-convoluting the IR and/or Raman spectroscopy obtained from the formulated product. This can be achieved by subtracting the known excipient signals from the IR and/or Raman spectroscopy of the formulated product. In addition, various chemometric techniques can be used to identify Compound 1 crystalline Form A hydrate in the composition, for example principal component analysis. Such analysis may be required to identify IR and/or Raman signals belonging to Compound 1 crystalline Form A hydrate as they may overlap with IR and/or Raman signals of some of the excipients used in the composition.

A summary of the characteristics of Compound 1 crystalline Form A hydrate is shown in Table 7.

TABLE 7
Characteristics of Compound 1 Crystalline Form A hydrate

| Compound 1 | | |
|---|---|---|
| Crystalline | | |
| Form A | Analytical | |
| hydrate | Technique | Results |
| | XRPD | Crystalline |
| | DSC | Endotherm 78.0° C., melt with decomp |
| | | 279-281° C. |
| | TG | 2.6% weight loss from start to 70° C. |
| | DVS | Slightly hygroscopic |
| | ¹H NMR in | Consistent with structure with 0.03 mol |
| | in DMSO-d6 | DMF and 0.03 mol hexane |
| | KF | Water content of 2.4% |
| | IR | Consistent with structure with water present |
| | Raman | Consistent with structure |

### B. Compound 1, Polymorph Crystalline Form B DMF Solvate

¹H NMR analysis in in DMSO-d6 of Compound 1 crystalline Form B DMF solvate (FIG. 12) is consistent with Compound 1 chemical structure and 1.6 moles of DMF per molecules of Compound 1. The ¹H NMR spectrum in DMSO-d6 of Compound 1 crystalline Form B DMF solvate is shown in FIG. 12. ¹H NMR in DMSO-d6 peak list is shown in Table 8.

TABLE 8
¹H NMR peaks for Compound 1 Crystalline Form B DMF solvate
Chemical Shift (ppm)

| | |
|---|---|
| 3.58 | (s) |
| 4.05 | (m) |
| 4.38 | (m) |
| 4.65 | (dd) |
| 4.91 | (t) |
| 7.07 | (t) |
| 7.22 | (t) |
| 7.29 | (s) |
| 7.43 | (d) |
| 7.48 | (t) |
| 7.60 | (dd) |
| 7.68 | (d) |
| 7.74 | (t) |
| 7.90 | (dd) |
| 7.95 | (s) |
| 8.10 | (s) |
| 8.20 | (dd) |
| 8.25 | (s) |
| 10.19 | (s) |
| 11.73 | (d) |
| 11.84 | (d) |

Compound 1 crystalline Form B DMF solvate was characterized by the XRPD pattern shown in FIG. 9. Compound 1 crystalline Form B DMF solvate is a crystalline DMF solvate. The XRPD pattern of Compound 1 crystalline Form B DMF solvate is expressed in terms of the degree (20) and relative intensities, measured on a Rigaku SmartLab X-Ray Diffractometer. Table 9 shows the XRPD analysis of Compound 1 crystalline Form B DMF solvate, with select peaks unique to Compound 1 crystalline Form B DMF solvate shown in bold.

TABLE 9
XRPD full peak listing and relative intensities of Compound 1 Crystalline
Form B DMF solvate

| | Relative | d-spacing |
|---|---|---|
| °2θ ± 0.2 | Intensity | value (Å) |
| 4.5 | 63.82 | 19.8119 |
| 7.3 | 33.88 | 12.1760 |
| 7.7 | 98.40 | 11.4515 |
| 9.2 | 23.48 | 9.5708 |
| 10.3 | 18.34 | 8.5881 |
| 12.6 | 20.62 | 7.0474 |
| 12.8 | 16.46 | 6.9158 |
| 13.3 | 12.33 | 6.6371 |
| 14.3 | 86.18 | 6.1850 |
| 14.5 | 38.36 | 6.1002 |
| 14.7 | 18.85 | 6.0259 |
| 15.4 | 15.04 | 5.7388 |
| 16.0 | 13.75 | 5.5254 |
| 16.4 | 50.21 | 5.3919 |
| 16.8 | 27.32 | 5.2709 |
| 17.7 | 19.91 | 5.0220 |
| 18.5 | 24.82 | 4.7907 |
| 19.6 | 42.14 | 4.5291 |
| 20.7 | 21.99 | 4.2827 |
| 21.1 | 14.32 | 4.2183 |
| 21.7 | 42.80 | 4.0991 |
| 23.2 | 11.32 | 3.8338 |
| 24.2 | 19.36 | 3.6776 |
| 24.5 | 100.00 | 3.6274 |
| 24.9 | 46.86 | 3.5786 |
| 25.8 | 46.95 | 3.4583 |
| 26.3 | 32.76 | 3.3911 |
| 27.1 | 17.91 | 3.2879 |
| 27.7 | 14.41 | 3.2227 |
| 27.9 | 11.66 | 3.1955 |
| 28.5 | 29.72 | 3.1296 |
| 29.1 | 13.59 | 3.0644 |
| 29.4 | 18.88 | 3.0339 |
| 29.7 | 13.25 | 3.0099 |
| 30.4 | 14.53 | 2.9402 |
| 31.2 | 15.10 | 2.8631 |
| 32.4 | 8.98 | 2.7615 |
| 33.2 | 10.14 | 2.6984 |
| 35.6 | 11.98 | 2.5232 |
| 36.1 | 9.64 | 2.4907 |
| 36.6 | 10.27 | 2.4565 |
| 36.9 | 10.63 | 2.4346 |
| 37.3 | 10.36 | 2.4119 |
| 38.4 | 12.20 | 2.3465 |
| 39.9 | 13.84 | 2.2616 |

For solid compositions comprising Compound 1 crystalline Form B DMF solvate, the solid compositions can be analyzed by XRPD to identify Compound 1 crystalline Form B DMF solvate in the product. This can be done by deconvoluting the XRPD data obtained from the formulated product. This can be achieved by subtracting the known excipient signals from the XRPD data of the formulated product. In addition, various chemometric techniques can be used to identify Compound 1 crystalline Form B DMF solvate in the solid composition, for example principal component analysis. Such analysis may be required to identify XRPD peaks belonging to Compound 1 crystalline Form B DMF solvate as they may overlap with XRPD peaks of some of the excipients used in the composition.

The DSC thermogram of Compound 1 crystalline Form B DMF solvate (FIG. 10) exhibits a broad endotherm at 147° C.

The TG thermogram of Compound 1 crystalline Form B DMF solvate (FIG. 11) exhibits a weight loss of 18.3%, up to 185° C.

Additionally, indexing of Compound 1 crystalline Form B DMF solvate was successful, suggesting a pure crystalline phase. Based on volume consideration, Compound 1 crystalline Form B DMF solvate was determined to be a solvate.

A summary of the characteristics of Compound 1 crystalline Form B DMF solvate is shown in Table 10.

TABLE 10
Summary of characterization data for Compound
1 Crystalline Form B DMF solvate

| Compound 1 | | |
|---|---|---|
| Crystalline | | |
| Form B | Analytical | |
| DMF solvate | Technique | Results |
| | XRPD | Crystalline |
| | DSC | Endotherm 147° C. |
| | TG | 18.3% weight loss from start to 185° C. |
| | ¹H NMR in | Consistent with structure with 1.6 mol DMF |
| | DMSO-d6 | |

### C. Compound 1, Polymorph Crystalline Form C Anhydrate

Compound 1 crystalline Form C anhydrate is an anhydrous crystalline material. The XRPD diffractogram for Compound 1 crystalline Form C anhydrate is shown in FIG. 13.

The DSC thermogram of Compound 1 crystalline Form C anhydrate (FIG. 14) exhibits a broad endotherm at 100.55° C.

The TG thermogram of Compound 1 crystalline Form C anhydrate (FIG. 15) shows a minimal weight loss of 0.4% up to 135° C.

The DVS data for Compound 1 crystalline Form C anhydrate (FIG. 16) shows a weight gain of 0.35% between 5 and 95% RH.

Indexing of Compound 1 crystalline Form C anhydrate was successful, indicating it is a pure crystalline phase. Acetonitrile was not part of the crystal lattice based on volume consideration.

The ¹H NMR spectrum in DMSO-d6 (FIG. 19) of Compound 1 crystalline Form C anhydrate is consistent with Compound 1 chemical structure and 0.07 moles of acetonitrile (ACN) per mole of Compound 1, which corresponds to the weight loss observed from the TG data. ¹H NMR in DMSO-d6 peak list is shown in Table 11.

TABLE 11
¹H NMR peaks for Compound 1 Crystalline Form C anhydrate
Chemical Shift (ppm)

| | |
|---|---|
| 3.58 | (s) |
| 4.05 | (m) |
| 4.38 | (m) |
| 4.65 | (dd) |
| 4.91 | (t) |
| 7.07 | (t) |
| 7.22 | (t) |
| 7.29 | (s) |
| 7.43 | (d) |
| 7.49 | (t) |
| 7.60 | (dd) |
| 7.68 | (d) |
| 7.74 | (t) |
| 7.90 | (dd) |
| 8.09 | (s) |
| 8.20 | (dd) |
| 8.25 | (s) |
| 10.19 | (s) |
| 11.73 | (d) |
| 11.84 | (d) |

Compound 1 crystalline Form C anhydrate was characterized by the XRPD pattern shown in FIG. 13. The XRPD pattern of Compound 1 crystalline Form C anhydrate, is expressed in terms of the degree (2θ) and relative intensities, measured on a Rigaku SmartLab X-Ray Diffractometer. Table 12 shows the XRPD analysis of Compound 1 crystalline Form C anhydrate, with select peaks unique to Compound 1 crystalline Form C anhydrate shown in bold.

TABLE 12
XRPD full peak listing and relative intensities
of Compound 1 Crystalline Form C anhydrate

| | Relative | d-spacing |
|---|---|---|
| °2θ ± 0.2 | Intensity | value (Å) |
| 5.8 | 10.31 | 15.1850 |
| 9.8 | 27.97 | 9.0620 |
| 10.3 | 6.58 | 8.6215 |
| 11.7 | 16.42 | 7.5893 |
| 12.0 | 74.14 | 7.3873 |
| 12.9 | 7.62 | 6.8519 |
| 13.4 | 17.55 | 6.6075 |
| 14.3 | 47.57 | 6.1936 |
| 14.6 | 28.30 | 6.0505 |
| 15.2 | 82.32 | 5.8212 |
| 15.7 | 5.18 | 5.6443 |
| 16.7 | 5.46 | 5.2959 |
| 17.7 | 12.15 | 5.0052 |
| 17.9 | 14.02 | 4.9663 |
| 18.7 | 23.95 | 4.7551 |
| 19.4 | 13.74 | 4.5707 |
| 20.3 | 17.28 | 4.3702 |
| 20.6 | 25.28 | 4.3156 |
| 20.7 | 25.69 | 4.2950 |
| 21.0 | 24.38 | 4.2382 |
| 21.1 | 28.24 | 4.2065 |
| 21.6 | 13.85 | 4.1178 |
| 21.8 | 12.05 | 4.0805 |
| 22.1 | 100.00 | 4.0257 |
| 22.6 | 19.61 | 3.9308 |
| 23.1 | 24.76 | 3.8469 |
| 23.4 | 63.64 | 3.8015 |
| 24.7 | 8.33 | 3.6072 |
| 25.2 | 7.28 | 3.5339 |
| 25.5 | 36.61 | 3.4876 |
| 26.1 | 24.73 | 3.4115 |
| 26.5 | 25.01 | 3.3684 |
| 27.0 | 7.20 | 3.3071 |
| 27.2 | 9.10 | 3.2761 |
| 27.4 | 10.08 | 3.2596 |
| 27.6 | 25.42 | 3.2272 |
| 28.1 | 6.12 | 3.1799 |
| 28.7 | 21.31 | 3.1083 |
| 29.5 | 14.99 | 3.0239 |
| 29.9 | 7.70 | 2.9844 |
| 30.7 | 7.12 | 2.9122 |
| 31.2 | 10.98 | 2.8684 |
| 32.3 | 4.74 | 2.7748 |
| 32.4 | 6.39 | 2.7599 |
| 32.8 | 4.36 | 2.7288 |
| 33.3 | 5.88 | 2.6937 |
| 33.5 | 4.43 | 2.6749 |
| 33.7 | 6.56 | 2.6595 |
| 34.3 | 3.99 | 2.6143 |
| 34.5 | 3.81 | 2.5967 |
| 35.0 | 5.57 | 2.5665 |
| 35.5 | 6.00 | 2.5314 |
| 35.9 | 5.69 | 2.5001 |
| 36.5 | 4.57 | 2.4603 |
| 37.3 | 5.77 | 2.4082 |

For solid compositions comprising Compound 1 crystalline Form C, the solid compositions can be analyzed by XRPD to identify Compound 1 crystalline Form C anhydrate in the product. This can be achieved by deconvoluting the XRPD data obtained from the formulated product. This can be achieved by subtracting the known excipient signals from the XRPD data of the formulated product. In addition, various chemometric techniques can be used to identify Compound 1 crystalline Form C anhydrate in the solid composition, for example principal component analysis. Such analysis may be required to identify XRPD peaks belonging to Compound 1 crystalline Form C anhydrate as they may overlap with XRPD peaks of some of the excipients used in the composition.

The infrared (FIG. 17) and Raman (FIG. 18) spectra are consistent with the chemical structure of Compound 1. Tables 13 and 14 show IR peaks and Raman shifts, respectively, with select peaks/shifts unique to Compound 1 crystalline Form C anhydrate shown in bold.

TABLE 13
Infrared peak list of Compound 1 Crystalline Form C anhydrate
IR Peak (cm⁻¹) ± 2

408
436
453
473
495
519
533
554
570
585
585
604
612
628
641
668
683
703
714
731
749
77
798
814
820
882
897
910
930
957
986
1007
1034
1048
1086
1124
1141
1150
1182
1231
1245
1274
1308
1336
1402
1421
1462
1474
1514
1539
1582
1606
1616
1648
3247
3310
3350

TABLE 14
Complete Raman shift list for Compound 1 Crystalline Form C anhydrate
Raman shift (cm⁻¹) ± 2

176
190
224
253
309
344
37
409
435
473
521
533
554
573
608
637
668
714
728
753
797
811
830
843
882
897
913
957
988
1005
1032
1086
1117
1140
1233
1256
1273
1306
1343
1376
1399
1426
1447
1472
1513
1584
1623
1651
2888
2940
2974
3001
3053
3082
3160
3250
3354

For compositions comprising Compound 1 crystalline Form C anhydrate, the compositions can be analyzed by IR and/or Raman spectroscopy to identify Compound 1 crystalline Form C anhydrate in the product. This can be achieved by deconvoluting the IR and/or Raman spectroscopy obtained from the composition. This can be achieved by subtracting the known excipient signals from the IR and/or Raman spectroscopy of the crystalline Formulated product. In addition, various chemometric techniques can be used to identify Compound 1 crystalline Form C anhydrate in the product, for example principal component analysis. Such analysis may be required to identify IR and/or Raman signals belonging to Compound 1 crystalline Form C anhydrate as they may overlap with IR and/or Raman signals of some of the excipients used in the composition.

A summary of the characteristics of Compound 1 crystalline Form C anhydrate is shown in Table 15.

TABLE 15
Summary of characterization data for Compound
1 Crystalline Form C anhydrate

| Compound 1 | | |
|---|---|---|
| Crystalline | | |
| Form C | Analytical | |
| anhydrate | Technique | Results |
| | XRPD | Crystalline |
| | DSC | Endo 100.55° C. |
| | TG | 0.4% weight loss from start to 135° C. |
| | ¹H NMR in | Consistent with structure with 0.07 mol ACN |
| | DMSO-d6 | |
| | DVS | Weight gain of 0.35% at 5-95% RH |
| | IR | Consistent with structure |
| | Raman | Consistent with structure |

### D. Compound 1, Polymorph Crystalline Form E DMSO Solvate

Compound 1 crystalline Form E is crystalline and a DMSO solvate.

Indexing of Compound 1 crystalline Form E DMSO solvate was unsuccessful, suggesting that the material was not a pure crystalline phase. However, the XRPD pattern is suggestive of a solvate with a large amount of disordered scattering observed at 14-28° 2θ and no peaks observed beyond 30° 2θ.

Approximately 1.6 moles of DMSO per mole of Compound 1 crystalline Form E DMSO solvate were observed in the 1H NMR spectrum in DMSO-d6 (FIG. 21). Table 16 shows the ¹H NMR in DMSO-d6 peak list.

TABLE 16
¹H NMR peaks for Compound 1 Crystalline Form E DMSO solvate
Chemical Shift (ppm)

| | |
|---|---|
| 2.53 | (s) |
| 3.58 | (s) |
| 4.05 | (m) |
| 4.38 | (m) |
| 4.65 | (dd) |
| 4.91 | (t) |
| 7.07 | (t) |
| 7.22 | (t) |
| 7.29 | (s) |
| 7.43 | (d) |
| 7.48 | (t) |
| 7.60 | (dd) |
| 7.68 | (d) |
| 7.74 | (t) |
| 7.90 | (dd) |
| 8.09 | (s) |
| 8.20 | (dd) |
| 8.25 | (s) |
| 10.19 | (s) |
| 11.73 | (d) |
| 11.84 | (d) |

Compound 1 crystalline Form E DMSO solvate was characterized by the XRPD pattern shown in FIG. 20. The XRPD pattern of Compound 1 crystalline Form E DMSO solvate, is expressed in terms of the degree (20) and relative intensities, measured on a Rigaku SmartLab X-Ray Diffractometer. Table 17 shows the XRPD analysis of Compound 1 crystalline Form E DMSO solvate, with select peaks unique to Compound 1 crystalline Form E DMSO solvate shown in bold.

TABLE 17
XRPD full peak listing and relative intensities
of Compound 1 Crystalline Form E DMSO solvate

| | Relative | d-spacing |
|---|---|---|
| °2θ ± 0.2 | Intensity | value (Å) |
| 6.0 | 100.00 | 14.7791 |
| 6.5 | 74.44 | 13.6398 |
| 8.6 | 34.60 | 10.2578 |
| 9.1 | 32.68 | 9.7391 |
| 9.5 | 15.99 | 9.2705 |
| 11.9 | 46.16 | 7.4243 |
| 12.4 | 12.75 | 7.1495 |
| 12.9 | 27.99 | 6.8413 |
| 13.5 | 24.16 | 6.5684 |
| 13.7 | 16.75 | 6.4541 |
| 14.3 | 23.80 | 6.2109 |
| 14.9 | 19.03 | 5.9614 |
| 15.7 | 21.81 | 5.6372 |
| 16.4 | 16.34 | 5.4181 |
| 16.6 | 18.14 | 5.3467 |
| 17.0 | 16.86 | 5.2277 |
| 17.3 | 29.78 | 5.1375 |
| 17.5 | 24.48 | 5.0676 |
| 17.9 | 34.31 | 4.9443 |
| 18.8 | 19.62 | 4.7300 |
| 19.0 | 54.43 | 4.6659 |
| 19.4 | 21.53 | 4.5660 |
| 19.8 | 45.76 | 4.4838 |
| 20.3 | 27.97 | 4.3788 |
| 21.0 | 28.58 | 4.2382 |
| 21.3 | 26.49 | 4.1713 |
| 21.8 | 41.97 | 4.0694 |
| 22.1 | 49.23 | 4.0149 |
| 23.2 | 55.30 | 3.8306 |
| 24.0 | 78.06 | 3.7139 |
| 24.7 | 29.44 | 3.6014 |
| 25.0 | 48.92 | 3.5673 |
| 25.2 | 63.17 | 3.5339 |
| 25.8 | 33.28 | 3.4504 |
| 26.1 | 46.29 | 3.4115 |
| 27.1 | 33.08 | 3.2951 |
| 28.0 | 15.72 | 3.1888 |
| 28.5 | 17.30 | 3.1361 |
| 29.1 | 15.20 | 3.0644 |
| 29.7 | 20.03 | 3.0119 |
| 30.1 | 13.78 | 2.9650 |
| 30.7 | 12.25 | 2.9103 |
| 31.7 | 11.32 | 2.8226 |
| 32.7 | 11.57 | 2.7352 |
| 35.5 | 10.48 | 2.5287 |
| 36.9 | 9.69 | 2.4384 |
| 38.2 | 10.20 | 2.3536 |

For solid compositions comprising Compound 1 crystalline Form E DMSO solvate, the solid compositions can be analyzed by XRPD to identify Compound 1 crystalline Form E DMSO solvate in the solid composition. This can be achieved by deconvoluting the XRPD data obtained from the composition. This can be achieved by subtracting the known excipient signals from the XRPD data of the formulated product. In addition, various chemometric techniques can be used to identify Compound 1 crystalline Form E DMSO solvate in the composition, for example principal component analysis. Such analysis may be required to identify XRPD peaks belonging to Compound 1 crystalline Form E DMSO solvate as they may overlap with XRPD peaks of some of the excipients used in the composition.

A summary of the characteristics of Compound 1 crystalline Form E DMSO solvate is shown in Table 18.

TABLE 18
Summary of characterization data for Compound
1 Crystalline Form E DMSO solvate

| Compound 1 | | |
|---|---|---|
| Crystalline Form | | |
| E DMSO solvate | Analytical Technique | Results |
| | XRPD | Crystalline |
| | ¹H NMR in DMSO-d6 | Consistent with structure |
| | | with 1.6 mol DMSO |

### E. Compound 1, Polymorph Crystalline Form F DMA Solvate

Compound 1 crystalline Form F DMA solvate is a crystalline DMA solvate.

Compound 1 crystalline Form F DMA solvate was successfully indexed, suggesting a pure crystalline phase. Based on volume consideration, crystalline Form F DMA solvate was consistent to be a mono-DMA solvate.

The ¹H NMR result in DMSO-d6 (FIG. 23) shows 0.82 moles of DMA per molecules of Compound 1, consistent with the indexing result. Table 19 shows the 1H NMR in DMSO-d6 peak list.

TABLE 19
¹H NMR peaks for Compound 1 Crystalline Form F DMA solvate
Chemical Shift (ppm)

| | |
|---|---|
| 1.95 | (s) |
| 2.78 | (s) |
| 2.94 | (s) |
| 3.58 | (s) |
| 4.05 | (m) |
| 4.38 | (m) |
| 4.65 | (dd) |
| 4.91 | (t) |
| 7.07 | (t) |
| 7.22 | (t) |
| 7.29 | (s) |
| 7.43 | (d) |
| 7.48 | (t) |
| 7.60 | (dd) |
| 7.68 | (d) |
| 7.74 | (t) |
| 7.90 | (dd) |
| 8.09 | (s) |
| 8.20 | (dd) |
| 8.25 | (s) |
| 10.19 | (s) |
| 11.73 | (d) |
| 11.84 | (d) |

Compound 1 crystalline Form F DMA solvate was characterized by the XRPD pattern shown in FIG. 22. The XRPD pattern of Compound 1 crystalline Form F DMA solvate, is expressed in terms of the degree (2θ) and relative intensities, measured on a Rigaku SmartLab X-Ray Diffractometer. Table 20 shows the XRPD analysis of Compound 1 crystalline Form F DMA solvate, with select peaks unique to Compound 1 crystalline Form F DMA solvate shown in bold.

TABLE 20
XRPD full peak listing and relative intensities
of Compound 1 Crystalline Form F DMA solvate

| | Relative | d-spacing |
|---|---|---|
| °2θ ± 0.2 | Intensity | value (Å) |
| 7.8 | 19.83 | 11.3925 |
| 8.1 | 18.01 | 10.9421 |
| 9.1 | 35.49 | 9.7391 |
| 9.5 | 12.18 | 9.3487 |
| 9.9 | 27.47 | 8.9342 |
| 11.5 | 8.69 | 7.6812 |
| 12.8 | 20.65 | 6.9266 |
| 12.9 | 15.26 | 6.8413 |
| 14.8 | 7.72 | 5.9855 |
| 15.5 | 19.53 | 5.7021 |
| 16.0 | 22.05 | 5.5323 |
| 16.9 | 33.15 | 5.2461 |
| 17.6 | 8.65 | 5.0504 |
| 18.2 | 11.69 | 4.8742 |
| 18.5 | 14.39 | 4.7907 |
| 18.6 | 13.47 | 4.7602 |
| 19.8 | 7.85 | 4.4883 |
| 20.5 | 10.09 | 4.3239 |
| 21.3 | 6.42 | 4.1713 |
| 22.1 | 8.31 | 4.0293 |
| 22.7 | 16.33 | 3.9240 |
| 24.3 | 16.82 | 3.6627 |
| 25.5 | 100.00 | 3.4984 |
| 25.9 | 51.44 | 3.4348 |
| 26.7 | 10.19 | 3.3436 |
| 27.5 | 10.05 | 3.2480 |
| 29.5 | 7.17 | 3.0239 |

For solid compositions comprising Compound 1 crystalline Form F DMA solvate, the solid compositions can be analyzed by XRPD to identify Compound 1 crystalline Form F DMA solvate in the solid composition. This can be achieved by de-convoluting the XRPD data obtained from the composition. This can be achieved by subtracting the known excipient signals from the XRPD data of the formulated product. In addition, various chemometric techniques can be used to identify Compound 1 crystalline Form F DMA solvate in the composition, for example principal component analysis. Such analysis may be required to identify XRPD peaks belonging to Compound 1 crystalline Form F DMA solvate as they may overlap with XRPD peaks of some of the excipients used in the composition.

A summary of the characteristics of Compound 1 crystalline Form F DMA solvate is shown in Table 21.

TABLE 21
Summary of characterization data for Compound
1 Crystalline Form F DMA solvate

| Compound 1 | | |
|---|---|---|
| Crystalline | | |
| Form F DMA | Analytical | |
| solvate | Technique | Results |
| | XRPD | Crystalline |
| | ¹H NMR in | Consistent with structure with 0.82 |
| | DMSO-d6 | mol DMA |

C. Differential Scanning calorimetry (DSC)

The DSC analysis was carried out using a TA Instruments Q2500 Discovery Series instrument. The instrument temperature calibrations were performed using indium. The DSC cell was kept under a nitrogen purge of ~50 mL per minute during the analysis. The sample was placed in a standard, crimped aluminum pan and heated from approximately 25° C. to 250° C. at a rate of 10° C. per minute. Crystalline Form A hydrate was heated up to 350° C.

### D. Thermogravimetric Analysis (TGA)

The TG analysis was carried out using a TA Instruments Q50 Discovery Series instrument. The nitrogen purge was ~10 mL per minute at the balance and ~90 mL per minute at the furnace. The sample was placed into a pre-tared platinum pan and heated to approximately 25° C. to 350° C. at a rate of 10° C. per minute.

### E. Dynamic Vapor Sorption (DVS) Analysis

The DVS analysis was carried out using a TA Instruments Q5000 Dynamic Vapor Sorption analyzer. A sample weight of less than 10 mg was loaded into a metal-coated quartz pan for analysis. The sample was analyzed at 25° C. after equilibrated to 5% relative humidity (RH) in 10% RH steps from 5 to 95% RH (adsorption cycle) and from 95 to 5% RH (desorption cycle). The movement from one step to the next occurred either after satisfying the equilibrium criterion of 0.01% weight change in 5 minutes or, if the equilibrium criterion was not met, after 90 minutes. The percent weight change values are calculated using Microsoft Excel®.

### F. Infrared (IR) Spectroscopy

IR spectroscopic analysis was performed using a Thermo Scientific model iS50 Fourier-transform (FT) IR spectrophotometer equipped with a deuterated triglycine sulfate (DTGS) detector, a potassium bromide (KBr) beamsplitter, and a Polaris™ long-life IR source. A diamond attenuated total reflectance (ATR) sampling accessory with a spectral range of 4000 cm⁻¹ to 400 cm⁻¹ was used. Each spectrum was the result of 128 co-added scans acquired at 2 cm⁻¹ resolution. A single beam background scan of air was acquired before the sample scan, allowing presentation of the spectra in log 1/R units. Wavelength calibration was performed using polystyrene. OMNIC v9.11 software package (Thermo-Nicolet) was used to acquire, process, and evaluate the spectral data.

### G. Fourier-Transform (FT) Raman Spectroscopy

Raman spectroscopic analysis was performed using a Nicolet iS50 Raman module that was equipped with a 1064 nm near-infrared laser. The system was configured with an indium gallium arsenide (InGaAs) detector and a calcium fluoride (CaF₂) beamsplitter. Each sample was placed onto the automated XYZ stage and analyzed using a laser power that was adjusted to optimize the signal intensity while avoiding damage to the sample. Raman spectra were collected with 256 signal-averaged scans at a resolution of 2 cm⁻¹ over the spectral range from 3700 cm⁻¹ to 100 cm⁻¹. Data acquisition and processing were performed using OMNIC v9.11 software.

### H. ¹H Nuclear Magnetic Resonance (NMR) Spectroscopy

The ¹H NMR spectra were acquired on a Bruker Avance II 400 spectrometer. Samples were prepared by dissolving material in DMSO-d6. The solutions were placed into individual 5-mm NMR tubes for subsequent spectral acquisition. The temperature controlled (298K) ¹H NMR spectra acquired on the Avance II 400 utilized a 5-mm cryoprobe operating at an observing frequency of 400.18 MHz. Each spectrum was processed using TopSpin version 4.1.4 and referenced to the chemical shift of the residual DMSO-d6 (2.5 ppm) peak.

1. Compound 1 crystalline Form A hydrate.

2. The Compound 1 crystalline Form A hydrate according to claim 1, having an X-ray powder diffraction pattern substantially as shown in FIG. 2.

3. The Compound 1 crystalline Form A hydrate according to claim 1, further characterized by an X-ray powder diffraction pattern comprising a peak at each of diffraction angles (2θ) 6.1±0.2°, 8.7±0.2°, 9.7±0.2°, 13.7±0.2°, 13.9±0.2°, 19.4±0.2°, 23.4±0.2, and 25.4±0.2° and obtained using a Cu tube anode.

4. The Compound 1 crystalline Form A hydrate according to claim 1, further characterized by an X-ray powder diffraction pattern comprising a peak at each of diffraction angles (2θ) 6.1±0.2°, 8.7±0.2°, 9.7±0.2°, 13.7±0.2°, and 13.9±0.2° and obtained using a Cu tube anode.

5. The Compound 1 crystalline Form A hydrate according to claim 1, further characterized by an X-ray powder diffraction pattern comprising a peak at each of diffraction angles (2θ) 8.7±0.2°, 9.7±0.2°, and 13.9±0.2° and obtained using a Cu tube anode.

6. The Compound 1 crystalline Form A hydrate according to claim 1, having a differential scanning calorimetry thermogram (DSC) comprising an endothermic peak at about 78.0° C.

7. The Compound 1 crystalline Form A hydrate according to claim 1, having a differential scanning calorimetry thermogram (DSC) comprising a melting transition at about 279° C. to about 281° C.

8. The Compound 1 crystalline Form A hydrate according to claim 1, having a differential scanning calorimetry thermogram (DSC) substantially as shown in FIG. 3.

9. The Compound 1 crystalline Form A hydrate according to claim 1, having a thermogravimetric analysis (TGA) curve substantially as shown in FIG. 4.

10. The Compound 1 crystalline Form A hydrate according to claim 1, further characterized by an IR spectrum substantially depicted in FIG. 6.

11. The Compound 1 crystalline Form A hydrate according to claim 1, further characterized by an IR spectrum having an absorption peak at each of 1426±2 cm⁻¹, 1547±2 cm⁻¹, and 1629±2 cm⁻¹.

12. The Compound 1 crystalline Form A hydrate according to claim 1, further characterized by a Raman spectrum substantially depicted in FIG. 7.

13. The Compound 1 crystalline Form A hydrate according to claim 1, further characterized by a Raman spectrum having a Raman shift at each of 1407±2 cm⁻¹, 1443±2 cm⁻¹, 1540±2 cm⁻¹, 1578±2 cm⁻¹, and 1628±2 cm⁻¹.

14. The Compound 1 crystalline Form A hydrate according to claim 1, further characterized by a Raman spectrum having a Raman shift at each of 1407±2 cm⁻¹, 1540±2 cm⁻¹, and 1578±2 cm⁻¹.

15. The Compound 1 crystalline Form A hydrate according to claim 1 further characterized by a dynamic vapor sorption profile substantially as shown in FIG. 5.

16. (canceled)

17. (canceled)

18. The Compound 1 crystalline Form A hydrate according to claim 1, having a water content of about 2.4 wt % by Karl Fisher titration.

19. (canceled)

20. A composition comprising the Compound 1 crystalline Form A hydrate according to claim 1 and at least one pharmaceutically acceptable carrier or diluent.

21. (canceled)

22. The composition according to claim 20, wherein the composition is substantially free of Compound 1 crystalline Form C anhydrate.

23. (canceled)

24. (canceled)

25. The composition according to claim 20, comprising the Compound 1 crystalline Form A hydrate in an amount of at least about 1% by weight of the composition.

26. A method of making the Compound 1 crystalline Form A hydrate according to claim 1, the method comprising:
admixing about 95:about 5 methanol:water (v/v) and Compound 1 to form a slurry, and
stirring the slurry at about 40° C. for about two days,
wherein the slurry comprises the Compound 1 crystalline Form A hydrate.

27. The method of making the Compound 1 crystalline Form A hydrate according to claim 26, further comprising separating the crystalline Form A hydrate from the slurry and drying the separated crystalline Form A hydrate.

28. Compound 1 crystalline Form B DMF solvate.

29. (canceled)

30. (canceled)

31. The Compound 1 crystalline Form B DMF solvate according to claim 28, further characterized by an X-ray powder diffraction pattern comprising a peak at each of diffraction angles (2θ) 4.5±0.2°, 7.3±0.2°, and 10.3±0.2° and obtained using a Cu tube anode.

32.-37. (canceled)

38. A composition comprising the crystalline Form B DMF solvate according to claim 28 and at least one pharmaceutically acceptable carrier or diluent.

39.-41. (canceled)

42. The composition according to claim 38, comprising the Compound 1 crystalline Form B DMF solvate in an amount of at least about 1% by weight of the composition.

43. (canceled)

44. (canceled)

45. Compound 1 crystalline Form C anhydrate.

46. (canceled)

47. (canceled)

48. The Compound 1 crystalline Form C anhydrate according to claim 45, further characterized by an X-ray powder diffraction pattern comprising a peak at each of diffraction angles (2θ) 9.8±0.2°, 11.7±0.2°, and 15.2±0.2° and obtained using a Cu tube anode.

49.-58. (canceled)

59. A composition comprising the Compound 1 crystalline Form C anhydrate according to claim 45 and at least one pharmaceutically acceptable carrier or diluent.

60. (canceled)

61. The composition according to claim 59, wherein the composition is substantially free of Compound 1 crystalline Form A hydrate.

62. (canceled)

63. (canceled)

64. The composition according to claim 59, comprising the Compound 1 crystalline Form C anhydrate in an amount of at least about 1% by weight of the composition.

65.-70. (canceled)

71. Compound 1 crystalline Form E DMSO solvate.

72. (canceled)

73. (canceled)

74. The Compound 1 crystalline Form E DMSO solvate according to claim 71, further characterized by an X-ray powder diffraction pattern comprising a peak at each of diffraction angles (2θ) 6.0±0.2°, 6.5±0.2°, and 8.6±0.2° and obtained using a Cu tube anode.

75.-79. (canceled)

80. A composition comprising the crystalline Form E DMSO solvate according to claim 71 and at least one pharmaceutically acceptable carrier or diluent.

81.-83. (canceled)

84. The composition according to claim 80, comprising the Compound 1 crystalline Form E DMSO solvate in an amount of at least about 1% by weight of the composition.

85. Compound 1 crystalline Form F DMA solvate.

86. (canceled)

87. (canceled)

88. The Compound 1 crystalline Form F DMA solvate according to claim 85, further characterized by an X-ray powder diffraction pattern comprising a peak at each of diffraction angles (2θ) 8.1±0.2°, 9.1±0.2°, and 9.9±0.2° and obtained using a Cu tube anode.

89.-92. (canceled)

93. A composition comprising the crystalline Form F DMA solvate according to claim 85 and at least one pharmaceutically acceptable carrier or diluent.

94.-96. (canceled)

97. The composition according to claim 93, comprising the Compound 1 Form F DMA solvate in an amount of at least about 1% by weight of the composition.

98.-101. (canceled)

102. The Compound 1 crystalline Form A hydrate according to claim 1, wherein the Compound 1 crystalline Form A hydrate is substantially free of Compound 1 crystalline Form C anhydrate.

103. (canceled)

104. A method for treating cancer in a mammal in need thereof, comprising the step of—administering to the mammal an effective amount of Compound 1 crystalline Form A according to claim 1.

105. The method according to claim 104, wherein the cancer is a solid-tumor cancer.

106. The method according to claim 105, wherein the solid-tumor cancer is fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon cancer, colorectal cancer, kidney cancer, pancreatic cancer, bone cancer, breast cancer, ovarian cancer, prostate cancer, esophageal cancer, stomach cancer, oral cancer, nasal cancer, throat cancer, squamous cell carcinoma, basal cell carcinoma, adenocarcinoma, sweat gland carcinoma, sebaceous gland carcinoma, papillary carcinoma, a papillary adenocarcinoma, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, uterine cancer, testicular cancer, small cell lung carcinoma, bladder carcinoma, lung cancer, epithelial carcinoma, glioma, glioblastoma multiforme, astrocytoma, medulloblastoma, craniopharyngioma, ependymoma, pinealoma, hemangioblastoma, acoustic neuroma, oligodendroglioma, meningioma, skin cancer, melanoma, neuroblastoma, retinoblastoma, or hepatocellular carcinoma.

107. The method according to claim 104, wherein the cancer is a blood cancer.

108. The method according to claim 107, wherein the blood cancer is leukemia, lymphoma, or myeloma.

109. The method according to claim 108, wherein the blood cancer is leukemia, and the leukemia is acute leukemia or a chronic leukemia.

110. The method of claim 108, wherein the blood cancer is leukemia, and the leukemia is lymphoblastic leukemia, myelogenous leukemia, lymphocytic leukemia, myelocytic leukemia, acute lymphoblastic B-cell leukemia, acute lymphoblastic T-cell leukemia, acute myeloblastic leukemia, acute promyelocytic leukemia, acute monoblastic leukemia, acute erythroleukemic leukemia, acute megakaryoblastic leukemia, acute myelomonocytic leukemia, acute nonlymphocyctic leukemia, acute undifferentiated leukemia, chronic myelocytic leukemia, chronic lymphocytic leukemia, or hairy cell leukemia.

111. The method of claim 108, wherein the blood cancer is lymphoma, and the lymphoma is Hodgkin's disease, non-Hodgkin's lymphoma, Waldenstrom's macroglobulinemia, heavy chain disease, or polycythemia vera.

112. The method of claim 108, wherein the blood cancer is myeloma, and the myeloma is solitary plasmacytoma, extramedullary plasmacytoma or multiple myeloma.
